(19) United States
(12) Patent Application Publication
(10) Pub. No.: US 2025/0057660 A1
(43) Pub. Date: Feb. 20, 2025

(54) Glenoid Implant

(71) Applicant: Steven C. Chudik, Western Springs, IL (US)

(72) Inventor: Steven C. Chudik, Western Springs, IL (US)

(21) Appl. No.: 18/805,440

(22) Filed: Aug. 14, 2024

Related U.S. Application Data
(60) Provisional application No. 63/532,703, filed on Aug. 15, 2023.

(51) Int. Cl. A61F 2/40; A61F 2/30

(52) U.S. Cl. CPC A61F 2/4081; A61F 2/30749; A61F 2002/30327; A61F 2002/30354; A61F 2002/30606; A61F 2002/30784; A61F 2002/3085; A61F 2002/30892

(57) ABSTRACT

A glenoid implant is disclosed having a bone fixation portion, a coupling portion, and an articulating portion. The bone fixation portion has a first socket. The articulating portion has a second socket on the rear side. The coupling portion is engaged with the second socket of the articulating portion and the coupling portion is engaged with the bone fixation portion in the first socket to form a connection between the bone fixation portion and the articulating portion.

This application claims the benefit of U.S. patent application No. 63/532,703, filed Aug. 15, 2023, which is hereby incorporated by reference in its entirety.

### FIELD OF THE INVENTION

This invention relates to glenoid implants.

### BACKGROUND OF THE INVENTION

Orthopaedic surgeons often perform joint replacement surgery on patients who suffer pain and physical limitations caused by joint surfaces which have degenerative, traumatic, or other pathologic damage. The success of these surgeries is directly related to the degree of morbidity associated with the surgical technique which is used, and also to the ability of the surgery to restore the natural anatomy and biomechanics of the joint. The present inventor recognized the need for improved glenoid implants and surgical outcomes.

### BRIEF SUMMARY OF THE INVENTION

A glenoid implant is disclosed. In some embodiments, the implant comprises a bone fixation portion, an articulating portion, and a coupling portion.

In some embodiments, the bone fixation portion has a first side, an articular side, a first protruding element on the first side, and a front surface on the articular side. The bone fixation portion defines a first socket open on the articular side adjacent the front surface. The first protruding element is arranged to engage a bone surface inside a glenoid cavity. The articulating portion has a wear-resistant articulating surface arranged for sliding contact upon an articulating humeral portion of a humerus, a rear side, a second socket open on the rear side, and a rear surface adjacent the second socket. The coupling portion is arranged to connect the bone fixation portion and the articulating portion. The coupling portion is engaged with the second socket of the articulating portion and the coupling portion is engaged with the bone fixation portion in the first socket to form a stable long-lasting but disengageable connection between the bone fixation portion and the articulating portion. The front surface of the bone fixation portion is behind the rear surface of the articulating portion.

In some embodiments, the coupling portion has a second protruding element on a second side of the coupling portion and a first portion on a first side of the coupling portion. The first portion is engaged with the second socket of the articulating portion and the second protruding element is engaged with the bone fixation portion in the first socket to form the stable long-lasting but disengageable connection between the bone fixation portion and the articulating portion.

In some embodiments, the coupling portion is a post. In some embodiments, the first socket is a first cannulation extending from a first aperture on the first protruding element on the first side to a second aperture on the articular side. The post is configured to engage the bone fixation portion at the first cannulation.

In some embodiments, the bone fixation portion defines a first cannulation extending from a first aperture on the articular side to a second aperture on a first protruding element on a first side. A second protruding element of the articulating portion is engaged with the coupling portion in a recess of the coupling portion and a protruding element of the coupling portion is engaged with the bone fixation portion in the first cannulation to form a stable long-lasting but disengageable connection between the bone fixation and articulating portions.

In some embodiments, the articulating portion comprises a second socket or coupling recess opposite of an articulating surface. The coupling portion comprises a first portion and a protruding element. The first portion is engaged with the articulating portion at the coupling recess and the protruding element of the coupling portion is engaged with the bone fixation portion in the first cannulation of the bone fixation portion to form a stable long-lasting but disengageable connection between the bone fixation and articulating portions.

In some embodiments, the glenoid implant comprises a bone fixation portion, an articulating portion, and a post. The post is configured to engage the articulating portion at a second cannulation of the articulating portion and the bone fixation portion at a first cannulation of the bone fixation portion to form a stable long-lasting but disengageable connection between the bone fixation portion and the articulating portions.

In some embodiments, the glenoid implant comprises a bone fixation portion, an articulating portion, a coupling portion, and a post. The post is engaged with the coupling portion in a second cannulation of the coupling portion and the bone fixation portion in a first cannulation of the bone fixation portion. The first portion of the coupling portion is engaged with the articulating portion in a second recess of the articulating portion and a protruding element of the coupling portion is engaged with the bone fixation portion in the first cannulation of the bone fixation portion to form a stable long-lasting but disengageable connection between the bone fixation and articulating portions.

In some embodiments, the post is a first post and the implant comprises a second post for connecting the articulating portion to the first post. The first post configured to engage a glenoid bone cavity.

A method of implanting a glenoid implant in a glenoid of a human is disclosed. A glenoid implant is implanted at a glenoid without dislocating the shoulder joint. In some embodiments, the implanting is done without transversely cutting the rotator cuff. In some embodiments, the humerus is distracted from the glenoid without dislocating the shoulder joint to assist with implantation.

A method of implanting a humeral implant in a proximal humerus of a human is disclosed. In some embodiments, the implanting is done without transversely cutting the rotator cuff. In some embodiments, the humerus is distracted from the glenoid without dislocating the shoulder joint to assist with implantation.

A trial component for a humeral or glenoid implant is disclosed. The trial components possess particular features which allow them to be more easily inserted and removed without dislocating the shoulder joint. In some embodiments, the trial component has an articular portion and a low-profile coupling portion. The articular portion has an articular surface. The low-profile coupling portion projects from the articular portion.

In some embodiments, the trial component has an articular portion having an articular surface and a back side. The articular portion forms a channel in the articular surface adjacent the backside and a recess in the back side. The channel connects to the recess. The channel and the recess are configured to receive a coupling component.

In some embodiments, the trial component has an articular portion and a protruding element. The articular portion has an articular surface. The protruding element extends from the articular portion. The protruding element comprises a ramp and a plateau. The ramp extends between a back of the articular portion and the plateau. In some embodiments, the articular portion has a second ramp that surrounds the articular surface.

A method of determining the appropriate size of a humeral implant or glenoid implant and a method of shoulder replacement is disclosed. A trial articular component is inserted or placed at the proximal humerus or at the glenoid without dislocating the shoulder joint. The shoulder joint is tested by moving the humerus. The trial articular component is removed. Thereafter an implant articular component may be implanted corresponding to the trial articular component or the steps of inserting, testing, removing are repeated until an appropriate sized or fitting trial articular component(s) is found. In some embodiments, the steps are performed without transversely cutting a rotator cuff. In some embodiments, the humerus is distracted from the glenoid without dislocating the shoulder joint before the step of inserting or placing and before the step of removing. The distraction is released for the step of testing.

Another method of determining the appropriate size of a humeral implant or glenoid implant and a method of shoulder replacement is disclosed. Exposed opposing surfaces of a glenoid and a humerus are aligned. A soft tissue bridging the shoulder joint is tensioned. A distance between the opposing surfaces is measured. Then an implant articular component of the humeral implant at the proximal humerus or of the glenoid implant at the glenoid may be implanted, without dislocating the shoulder joint, that corresponds to the distance between the opposing surfaces. This may be also performed without transversely cutting a rotator cuff.

In some embodiments, before implanting, a trial articular component is inserted or placed at the proximal humerus or at the glenoid without dislocating the shoulder joint, where the trial articular component corresponds to the measured distance between opposing surfaces in the shoulder joint. The shoulder joint is tested by moving the humerus. The trial articular component is removed. Thereafter an implant articular component may be implanted corresponding to the trial articular component or the steps of inserting, testing, removing are repeated until an appropriate sized or fitting trial articular component(s) is found. This may be also performed without transversely cutting a rotator cuff.

Numerous other features and advantages of the invention will be apparent to orthopaedic surgeons and other persons who are skilled in the art of shoulder repair and reconstruction, particularly after reviewing the following description of the embodiments of the present invention, the claims, and the accompanying drawings.

### BRIEF DESCRIPTION OF THE DRAWINGS

FIG. 1 is a perspective view of the principal members of a human glenohumeral anatomy, namely, the glenoid process of a scapula bone and the head of a humerus bone.

FIGS. 2A through 2C are perspective views, partially in section, of a humeral head engaging a traditional glenoid implant and illustrating the loosening complications in that engagement.

FIG. 3 is a perspective view of the new glenoid implant of the invention which is disclosed here having a wear-resistant articulating portion, an intermediate coupling portion and an implant bone fixation portion designed to reside within a glenoid vault.

FIG. 4 is an exploded view of the new implant shown in FIG. 3 arranged for assembly with the glenoid.

FIG. 4A is a front articular side perspective view of a bone fixation component of the implant of FIG. 4 with optional cannulation.

FIG. 4B is a front articular side view of another embodiment bone fixation component with optional cannulation usable with the implant of FIG. 4 and the other glenoid implants disclosed herein.

FIG. 5A is an enlarged cross-sectional view of the new implant's coupling portion engaged with some of the sides of a non-articulating protruding element of the new implant's articulating portion.

FIG. 5B is an enlarged cross-sectional view of the new implant's coupling portion engaged upon the non-articulating protruding element of the new implant's articulating portion.

FIG. 5C is an enlarged cross-sectional view of the new implant's coupling portion engaged with the non-articulating side of the new implant's articulating portion.

FIG. 5D is an enlarged cross-sectional view of the new implant's coupling portion engaged with the non-articulating protruding element and other segments of the non-articulating side of the new implant's articulating portion.

FIG. 5E is an enlarged cross-sectional view of the new implant's coupling portion engaged with an offset protruding element of the new implant's articulating portion.

FIG. 5F is an enlarged cross-sectional view of the new implant's coupling portion engaged with an asymmetric protruding element of the new implant's articulating portion.

FIG. 5G is an enlarged cross-sectional view of the new implant's coupling portion engaged with a plurality of protruding elements of the new implant's articulating portion.

FIG. 5H is an enlarged cross-sectional view of the new implant's coupling portion engaged with an irregularly shaped non-articulating protruding element of the new implant's articulating portion.

FIG. 5I is an enlarged cross-sectional view of the new implant's coupling portion engaged with and over a plurality of non-articulating elements of the new implant's articulating portion.

FIG. 5J is an enlarged cross-sectional view of the new implant's coupling portion disposed opposite to an irregularly shaped articulating portion of the new implant.

FIG. 5K is an enlarged cross-sectional view of the new implant's coupling portion with internal bridging reinforcements engaging a protruding element of the new implant's articulating portion.

FIG. 5L is an enlarged cross-sectional view of the new implant's coupling portion with internal surface irregularities engaging a protruding element of the new implant's articulating portion.

FIG. 6 is an exploded view of a configuration of the new implant which has multiple protruding elements instead of a single protruding element arranged for assembly with the glenoid.

FIG. 7 is an exploded view of an offset configuration of a protruding element of the new implant arranged for assembly with the glenoid.

FIG. 8 is an exploded view of an alternative configuration of the new implant's bone fixation portion to correct for abnormalities in glenoid version and tilt, arranged with the intermediate coupling portion and articulating portion for assembly with the glenoid.

FIG. 9 is an exploded view of another alternative configuration of the new implant's bone fixation portion to accommodate glenoid deformity, arranged with the intermediate coupling portion and articulating portion for assembly with the glenoid.

FIG. 10 is a perspective view of a bone fixation portion of the new implant disposed within a cavity in the glenoid.

FIG. 11 is a perspective view of a screw fixation of a bone fixation portion of the new implant within a cavity in the glenoid.

FIG. 12 is an exploded view of an alternative form of the new implant having a bone fixation portion, an intermediate coupling portion, and a convex articulating portion instead of a concave articulating portion.

FIG. 13A is a perspective view of the new implant with the convex articulating portion, fully assembled and installed in a cavity in the glenoid.

FIG. 13B is an enlarged sectional view of a segment of a protruding element on an articulating portion of the new implant shown in FIG. 13A assembled with a bone fixation portion of the implant inside the glenoid cavity.

FIG. 14 is an exploded view of the new implant where the intermediate portion is constructed of similar material to that of the articular portion which makes up the non-articulating surface of the articular portion.

FIG. 15 is a perspective view of the new implant with a convex articulating surface portion arranged to extend over an entire glenoid surface and having a coupling engagement element fixed off-center from a central axis of the articulating surface portion.

FIG. 16A is a perspective view of the new implant which has a convex articulating portion with an offset coupling element disposed in a cavity in the glenoid and part of the articulating surface portion extending outside the limits of the glenoid.

FIG. 16B is an enlarged sectional view of a segment of a protruding element on an articulating portion of the new implant shown in FIG. 16A assembled with a bone fixation portion of the implant inside the cavity in the glenoid.

FIG. 17A is an exploded view an alternative form of the new glenoid implant which includes a rimless bone fixation portion, a coupling portion and a concave articulating surface portion.

FIG. 17B is a perspective view of the new glenoid implant shown in FIG. 17A with a rimless bone fixation portion, a coupling and a concave articulating surface portion applied to a prepared surface in the glenoid.

FIG. 17C is an enlarged sectional view of the new glenoid implant with a rimless bone fixation portion, a coupling portion and a concave articulating portion assembled together.

FIG. 18A is an exploded view of the new glenoid implant with a rimless bone fixation portion, a coupling portion and a full concave articulating portion.

FIG. 18B is a perspective view of the new glenoid implant with a rimless bone fixation portion, a coupling portion and a full concave articulating portion applied to a prepared surface in the glenoid cavity.

FIG. 19A is an exploded view of the new glenoid implant with a rimless bone fixation portion, a coupling portion and a convex articulating portion.

FIG. 19B is a perspective view of the new glenoid implant with a rimless bone fixation portion, a coupling portion and a convex articulating portion applied to a prepared surface in the glenoid cavity.

FIG. 20A is an exploded side perspective view of another embodiment glenoid implant of the invention adjacent a glenoid.

FIG. 20B is a side view of another embodiment articulating portion useable in place of the articulating portion of FIG. 20A.

FIG. 21 is a side perspective view of the glenoid implant of FIG. 20 implanted in the glenoid.

FIG. 22 is an exploded side perspective view of another embodiment glenoid implant of the invention adjacent a glenoid.

FIG. 23 is a side perspective view of the glenoid implant of FIG. 22 in a stage of implantation.

FIG. 24A is a side perspective view of the glenoid implant of FIG. 22 implanted in the glenoid.

FIG. 24B is side view of another embodiment articulating portion usable with the glenoid implant of FIG. 22.

FIG. 24C is side view of another embodiment articulating portion usable with the glenoid implant of FIG. 22.

FIG. 25 is an exploded side perspective view of another embodiment glenoid implant of the invention adjacent a glenoid.

FIG. 26 is a side view of a second embodiment post usable with the glenoid implant of FIG. 25.

FIG. 27 is a side perspective view of the glenoid implant of FIG. 25 in a stage of implantation.

FIG. 28 is a side perspective view of the glenoid implant of FIG. 25 implanted in the glenoid.

FIG. 29 is an exploded side perspective view of another embodiment glenoid implant of the invention adjacent a glenoid.

FIG. 30 is a side perspective view of the glenoid implant of FIG. 29 in a stage of implantation.

FIG. 31 is a side perspective view of the glenoid implant of FIG. 29 implanted in the glenoid.

FIG. 32 is a side view of a trial humeral articular component and a humerus distracted from the glenoid without dislocating the shoulder.

FIG. 33 is a side perspective view of a trial glenoid articular component and a humerus distracted from the glenoid without dislocating the shoulder.

FIG. 34 is a side view of the trial glenoid articular component of FIG. 33 placed in a glenoid implant and engaged with a humeral implant where the distraction of the humerus is released.

FIG. 35 is a side perspective view of another embodiment trial humeral articular component and a humerus distracted from the glenoid without dislocating the shoulder.

FIG. 36 is a side view of another embodiment trial glenoid articular component and a humerus distracted from the glenoid without dislocating the shoulder.

FIG. 37 is a rear view of the trial glenoid articular component of FIG. 36.

FIG. 38 is a rear view of another embodiment trial glenoid articular component.

FIG. 39 is a side view of the trial glenoid articular component of FIG. 38.

FIG. 40 is a side view of the trial glenoid articular component of FIG. 36 placed in a glenoid implant and engaged with a humeral implant where the distraction of the humerus has been released.

FIG. 41 is a side view of another embodiment trial articular component for a glenoid or humeral implant.

FIG. 42 is a rear view of the trial articular component of FIG. 41.

FIG. 43 is a side view of another embodiment trial articular component for a glenoid or humeral implant.

FIG. 44 is a rear view of the trial articular component of FIG. 43.

FIG. 45 is a side view of another embodiment trial articular component for a glenoid or humeral implant.

FIG. 46 is a rear view of the trial articular component of FIG. 45.

FIG. 47 is a side view of another embodiment trial humeral articular component.

FIG. 48 is a side view of another embodiment trial glenoid articular component.

FIG. 49A is a side view of a measuring block.

FIG. 49B is a front view of a measuring block.

FIG. 50 is a side perspective view of a fork instrument engaged and holding together a trial humeral articular component and a trial glenoid articular component, and a humerus distracted from the glenoid without dislocating the shoulder.

FIG. 51 is a side perspective view a prepared humerus distracted from a prepared glenoid with another embodiment distractor.

FIG. 52 is a side perspective view a prepared humerus distracted from a prepared glenoid with another embodiment distractor.

FIG. 53 is a side perspective view of a paddle usable with the distractor of FIG. 51 or 52.

FIG. 54 is a side perspective view of a paddle usable with the distractor of FIG. 51 or 52.

FIG. 55 is a side perspective view of a paddle usable with the distractor of FIG. 51 or 52.

FIG. 56 is a side view of a second embodiment measurement device.

FIG. 57 is a side perspective view the second embodiment measuring device of FIG. 56 deployed at a prepared humerus and a prepared glenoid.

### DETAILED DESCRIPTION

The following description is presented to enable any person skilled in the art to make and use the invention. For the purposes of explanation, specific nomenclature is set forth to provide a plural understanding of the invention. While this invention is susceptible of embodiment in many different forms, this description describes and the drawings show specific embodiments of the invention with the understanding that the present disclosure is to be considered as an exemplification of the principles of the invention and is not intended to limit the invention to the specific embodiments illustrated.

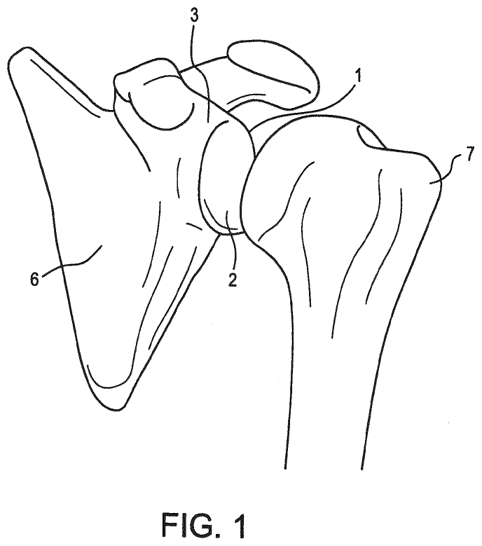

The new glenoid implants 30, 39, 51, 71, 90 which are disclosed here are directed to the repair of a human shoulder, the principal skeletal members of which are illustrated in FIG. 1. Seen there, the glenoid process 1 of a scapula bone 6 includes a glenoid articular surface 2 located over the glenoid vault bone 3. The upper end of a humerus bone 7 is situated opposite the surface 2.

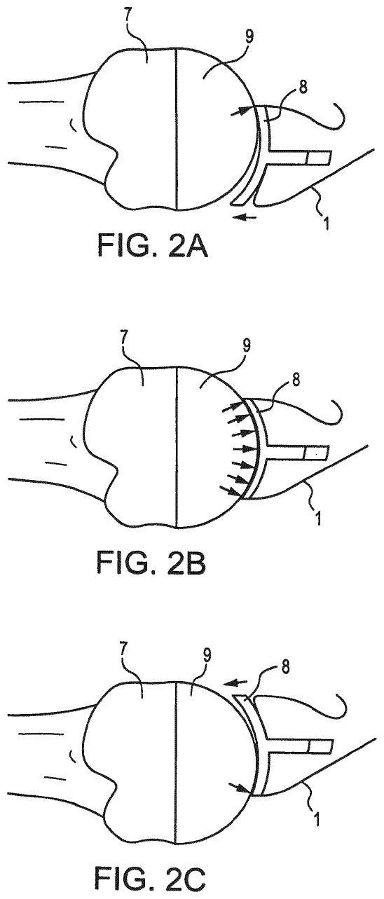

The standard treatment for repairing this joint, illustrated in FIGS. 2A-2C, often involves installing a standard glenoid implant 8 in the glenoid process 1 for another implant 9 on the head of the humerus to articulate against. However, as further illustrated by the several positions of the implants 8 and 9 in FIGS. 2A-2C, the glenoid implant 8 is pressured in one direction and then another as the humeral implant 9 articulates against it. Frequently the result is that the glenoid implant 8 becomes worn, or loosens, and requires replacement. Sometimes, too, repair of the joint requires a reversal of the cooperating articulating surfaces so that the glenoid implant's articulating face must be convex, rather than concave, and the humeral implant's articulating face must be the reverse, concave instead of convex.

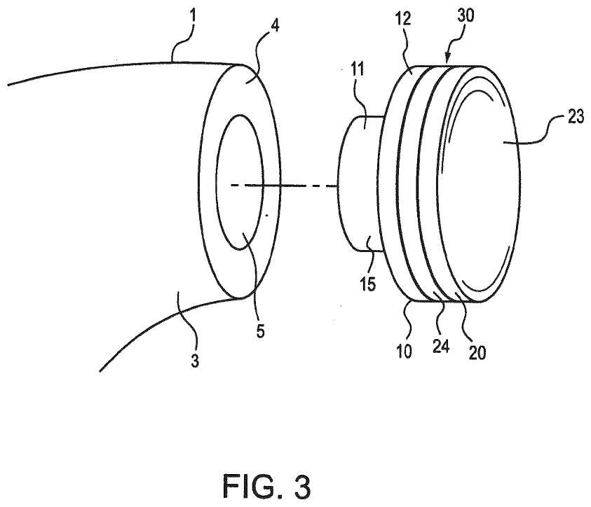

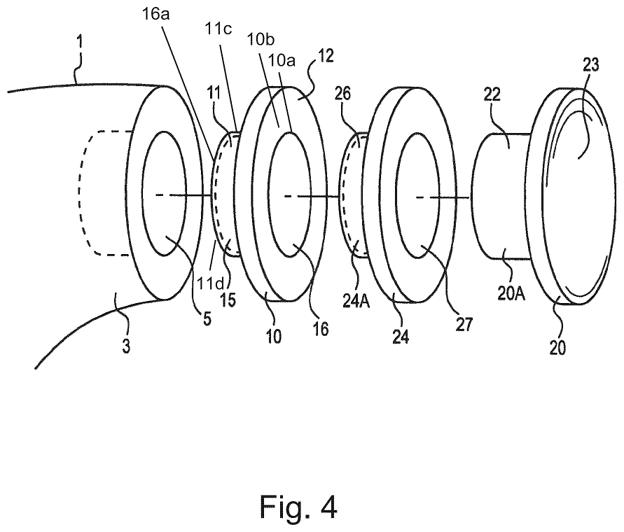

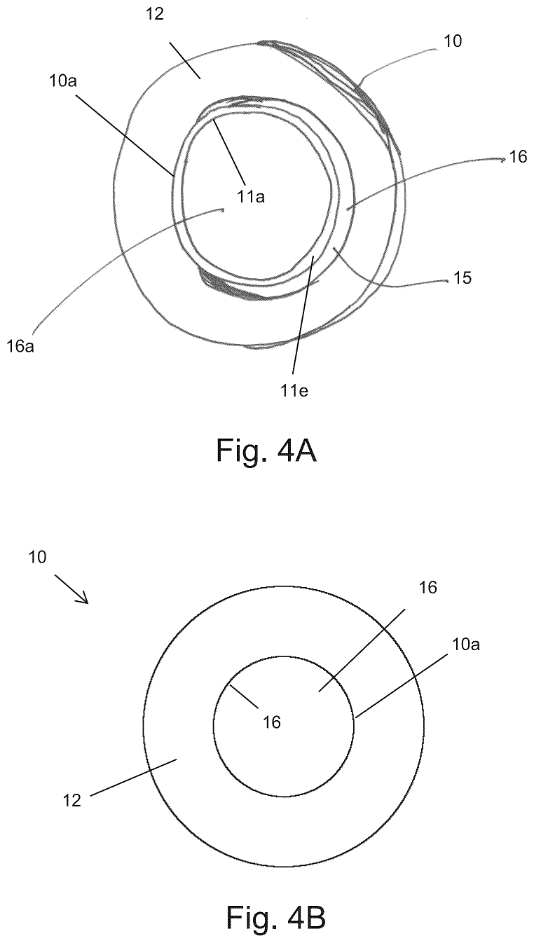

The new glenoid implant 30, an embodiment of which is illustrated in FIGS. 3 and 4, has three portions, namely, a bone fixation portion 10, an articulating portion 20, and a coupling portion 24 located intermediate the portions 10 and 20. The coupling portion, which engages both portions 10 and 20, forms a durable but disconnectable junction between the other two. The bone fixation portion 10 includes a hollow protruding element 11 with a bone ingrowth outer surface 15 for contacting and firmly joining a prepared surface of the glenoid inside the glenoid cavity. Also, on the inside wall of the protruding element 11, connecting surface 16 is arranged to engage coupling portion 24 on a connecting surface 26 located on the outside of coupling portion's 24 protruding element 24A.

In some embodiments, the bone fixation portion 10 comprises a cannulation 16a that extends from a first aperture 10a on the coupling side 10b through the bone fixation portion 10 to a second aperture 11a at the terminal end 11d of the protruding element 11. The cannulation extends through the interior of the protruding element 11. In some embodiments, the terminal end 11d of the protruding element 11 comprises a rim 11e surrounding and defining the second aperture 11a. In some embodiments, the terminal end 11d does not have a rim 11e and the cannulation comprises a uniform cross-section through the length of the bone fixation portion between the first aperture 10a on the articular side and the second aperture at the terminal end 11d of the protruding element 11 on the bone fixation side, such as shown in another embodiment bone fixation portion 10 in FIG. 4B. In some embodiments, the bone fixation portion 10 does not comprise a cannulation 16a and the protrusion houses a recess that is blind ended.

On the inside wall of protruding element 24A of the coupling portion 24, connecting surface 27 is arranged to engage the articulating portion 20 on a connecting surface 22 located on the outside of the articulating portion's 20 protruding element 20A.

The articulating portion 20 of the novel glenoid implant 30 includes either a relatively planar to slightly concave articulating surface element 23 for installation of the implant in an anatomic total shoulder arthroplasty or a convex articulating surface element 23A for installation of the implant in a reverse total shoulder arthroplasty.

The wall 11c of the protruding element 11 of the bone fixation portion 10 can possess a variable wall thickness such that the opposite surfaces, the bone ingrowth surface 15 and the connecting surface 16, may possess different dimensions and shapes.

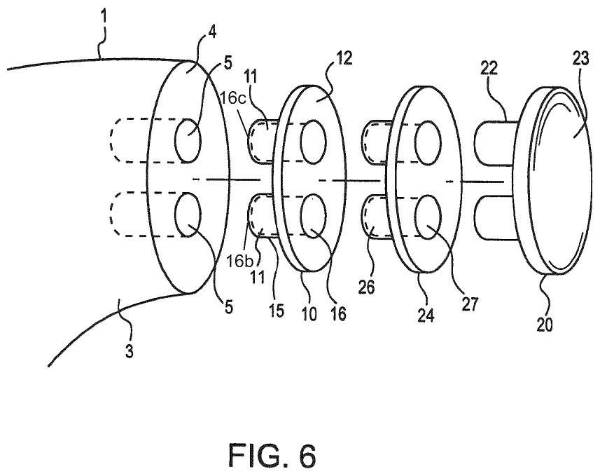

There may be a single protruding element 11 (FIG. 4) or multiple protruding elements 11 (FIG. 6). The bone fixation component may comprise a cannulation 16b, 16c, at one, a plurality, or all of the protruding elements of the embodiment having multiple protruding elements. The cannulations 16b, 16c are identical to the cannulation 16a of the embodiment of FIG. 4A or 4B, except for placement and size.

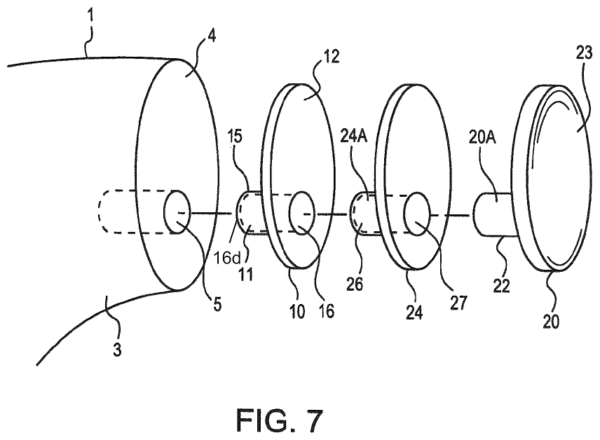

The protruding element 11 may also be offset (FIG. 7). The bone fixation component may comprise a cannulation 16d extending through the offset protruding element 11. The cannulations 16d is identical to the cannulation 16a, except for placement and size.

The connecting surface 16 of the bone fixation portion 10 may be configured as a “bore” (female portion) and the connecting surface 26 of the coupling portion 24, 24b, 24c may also be configured as a “trunnion” (male portion), composed of proper material with proper taper angle, diameter, length, conicity, surface finish, area of interference, and relative angle to allow a stable, long-lasting but disengageable “morse taper” connection between the bone fixation portion 10 and the articulating portion 20, 20b, 20d, 20e by means of the intermediate coupling portion 24, 24b, 24c. The connecting surfaces 16 and 26, as well as 22 and 27 may possess more than one trunnion and bore, mating geometry other than conical, or an additional asymmetric engaging geometric configuration to restrict rotational movement of the articular portion 20, 20b, 20d, 20e. A Morse taper connection provides a cone (male portion) in a cone (interior of female portion). The trunnion and bore are each tapered. When the bore driven onto the trunnion, or the trunnion is driven into the bore, or the trunnion is driven into the bore and the bore is driven onto the trunnion, the trunnion and bore come into intimate contact. The stresses on the materials of the bore and trunnion and the friction between keep the both components fixed together.

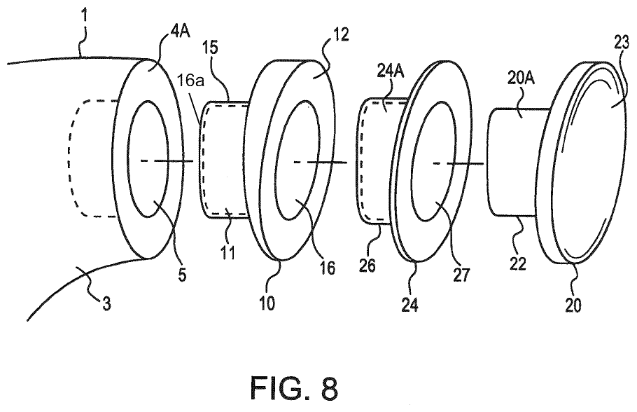

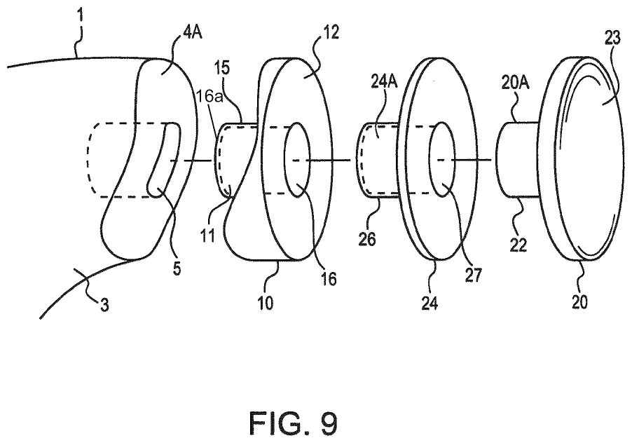

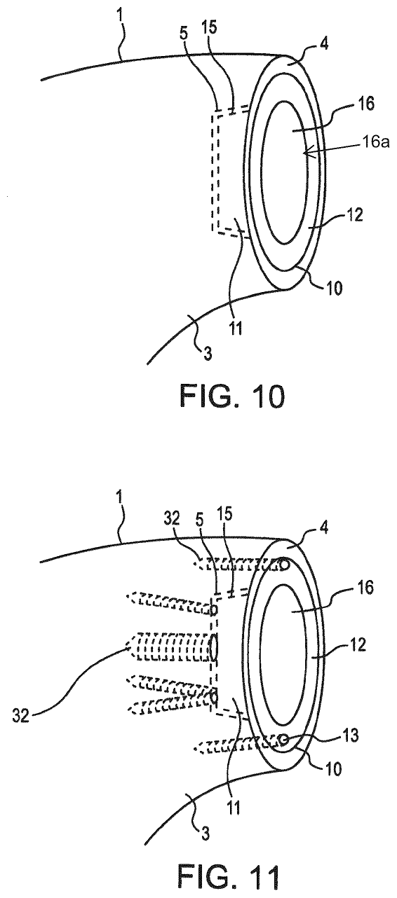

The bone fixation portion 10 is positioned both in the prepared geometric glenoid cavity 5 in the glenoid vault bone 3 (inlay) and on the prepared glenoid bone surface 4 (onlay) (FIG. 3). The bone fixation portion 10 is a shallow complete or incomplete shell with a protruding element 11 which extends into the glenoid vault bone 3 (FIG. 10). The protruding element 11 is surrounded by a complete or incomplete peripheral rim 12 which rests upon the prepared perimeter of glenoid bone 4 surrounding the prepared glenoid cavity 5 (FIG. 10). The thickness of the peripheral rim may be minimalized to maximize the allowable thickness for the articulating portion 20 while still providing a sufficiently stable base. The protruding element 11 of the bone fixation portion 10 protrudes into the glenoid 1 to a specified depth such that it is sufficient for the bone fixation portion 10 to be securely seated within the glenoid 1 and for the articulating portion 20 to fit therein with the intermediate coupling portion 24 while preserving as much glenoid bone 3 as possible. The bone fixation portion 10 may have different embodiments with or without a peripheral rim 12 or with variable geometric dimensions with optional screw cannulations 13 (FIG. 11). The bone ingrowth surface 15 of the protruding element 11 of the bone fixation portion 10 of the glenoid implant 30 can possess a convex dome, cylinder, cone, triangle, square, trapezoid, double cylinders, other complex geometric shape, or other multiple protruding dimensions for engagement in the prepared glenoid cavity 5. The cannulations 13 can be arranged to place screws 32 to add fixation strength (FIG. 11). The cannulations 13 can be limited to the protruding element 11 of the bone fixation portion 10 rather than the peripheral rim 12 to allow the rim 12 to be of minimal thickness. Alternatively, cannulations 13 can exist in either the peripheral rim 12 or in the protruding element 11 or both. Screws 32 can compress the bone fixation portion 10 into the glenoid bone cavity 5 and against the surface 4 and/or lock at a fixed angle and depth in the cannulations 13 of the bone fixation portion 10. The bone fixation portion 10 can be customized with variable peripheral rim 12 thickness and shape to fit an irregular or damaged glenoid surface 4A (FIGS. 8, 9).

The articulating surface portion 20 of the glenoid implant 30 has a smooth articulating surface 23 for contacting a humerus and uses the connecting surface 26 provided by the intermediate coupling portion 24 for disengageable connection to the connecting surface 16 of the bone fixation portion 10 (FIG. 4). The connecting surface 26 and intermediate coupling portion 24 may be of similar or different material than the articulating surface portion 20.

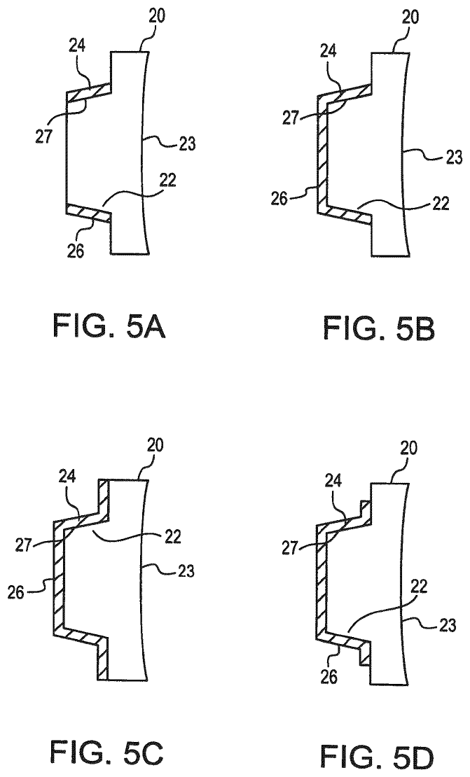

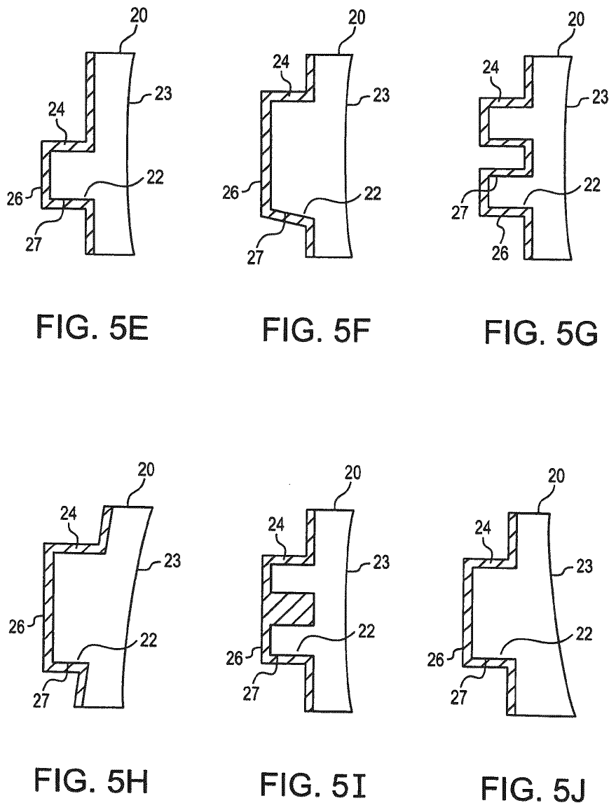

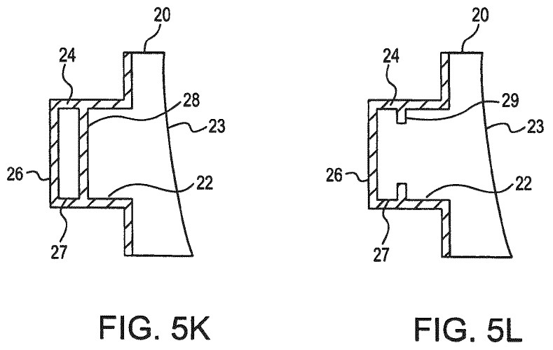

Connecting surface 26 is also configured to substantially conform to the connecting surface 16 of the bone fixation portion 10, including its protruding element 11 and peripheral rim 12. However, the connecting surface 26 of the intermediate coupling portion 24 may be continuous or discontinuous in a variety of configurations allowing for complete or incomplete contact with the connecting surface 16 of the bone fixation portion 10, and also allowing for a variety of configurations dictated by connecting surface 22 on the non-articulating side of the articulating portion 20 (FIGS. 5A-5L). The intermediate coupling portion 24 may also possess internal bridging reinforcements 28 or irregularities 29 of its connecting surface 27 to improve the connection with the articular portion 20 (FIGS. 5K, 5L). The planar or concave articulating surface portion 20 can be of variable thickness to correct glenoid version, tilt or other deformity (FIGS. 5E, 5H, 5J).

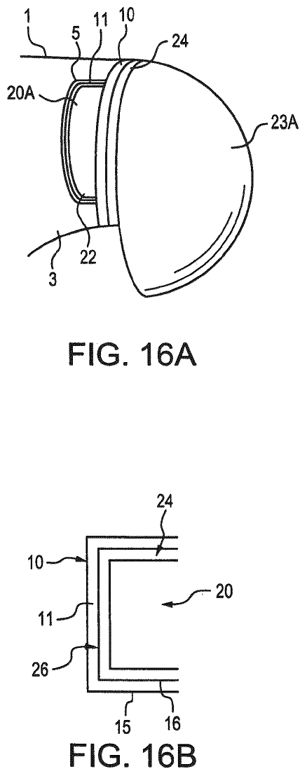

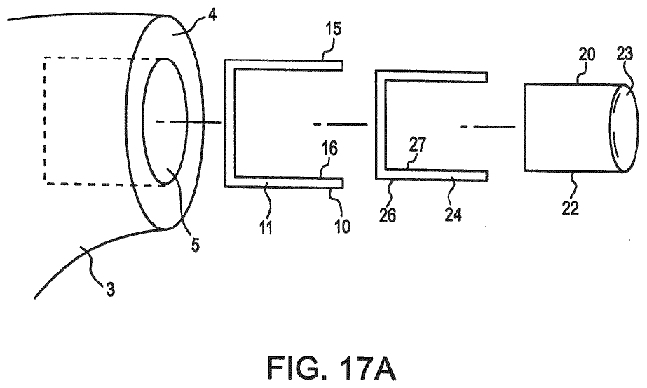

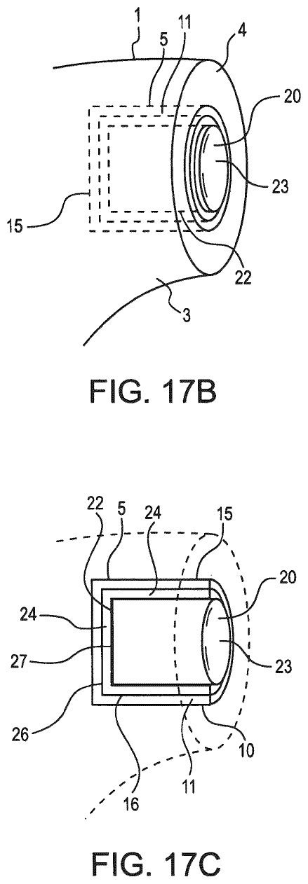

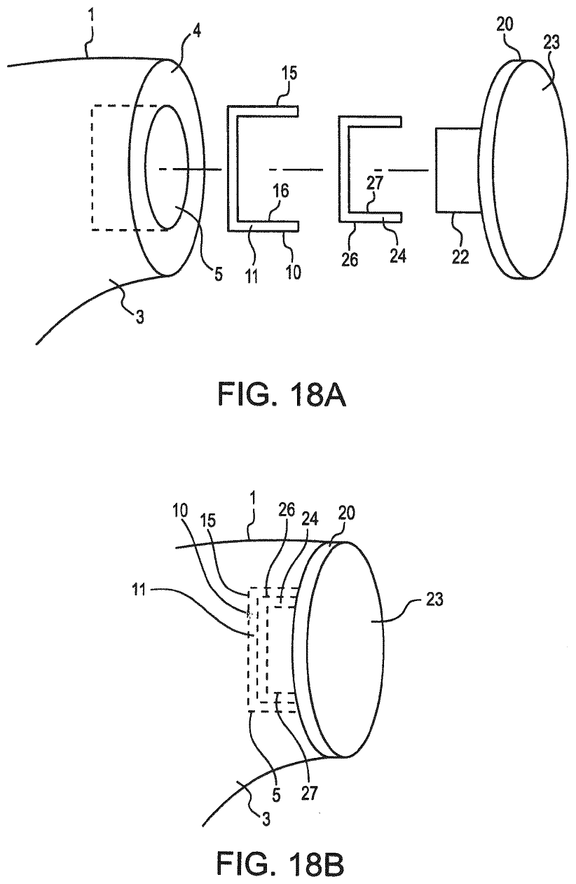

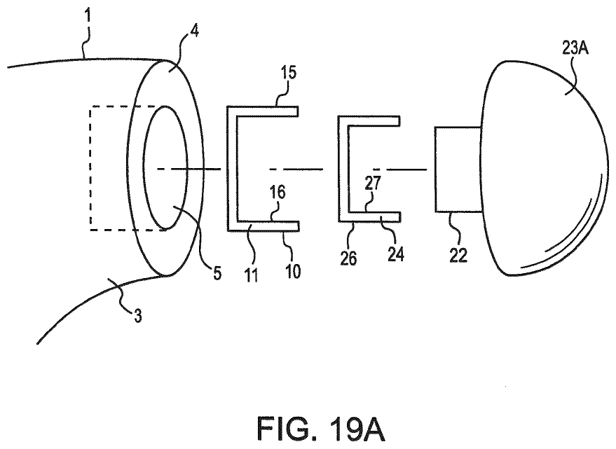

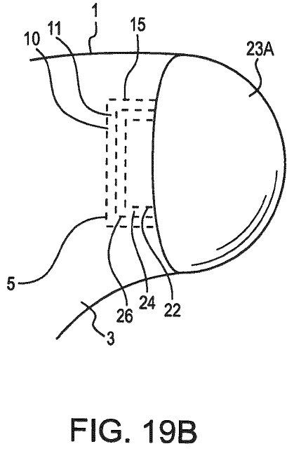

The protruding element 11 and its companion elements 24A and 20A may be offset from the centers of their respective portions, the bone fixation portion 10, the intermediate coupling portion 24, and/or the articulating portion 20 (FIGS. 7, 15, 16A). The bone fixation portion 10, intermediate coupling portion 24, and/or the articulating portion 20 may be rimless, without a peripheral rim (FIGS. 5A, 5B, 17A, 17B, 18A, 18B, 19A, 19B).

The glenoid articular surface 2 is prepared with a relatively planar reamer to mate with the peripheral onlay rim 12 of the bone fixation portion 10 of the glenoid implant 30. The central glenoid vault 3 is prepared by reaming, cutting and/or impacting it to create a geometric shaped cavity 5 within the glenoid 1. After inserting the protruding element 11 of bone fixation portion 10 into the glenoid cavity 5 and the peripheral rim 12 onto the prepared glenoid surface 4, screw(s) 32 can be inserted into optional cannulations 13 in the bone fixation portion 10 and the surrounding glenoid and scapular bone 1 to provide compressive and rigid locking fixation of the bone fixation portion 10 to the glenoid bone 1 (FIGS. 10, 11). The bone adherent and ingrowth surface 15 of the bone fixation portion 10 will provide a stable, long lasting connection. Any of the screws 32 can act as a stable fixed angle post to provide rigid positioning of the bone fixation portion 10.

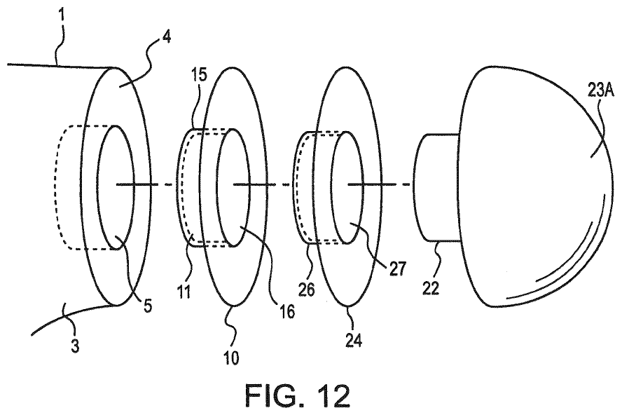

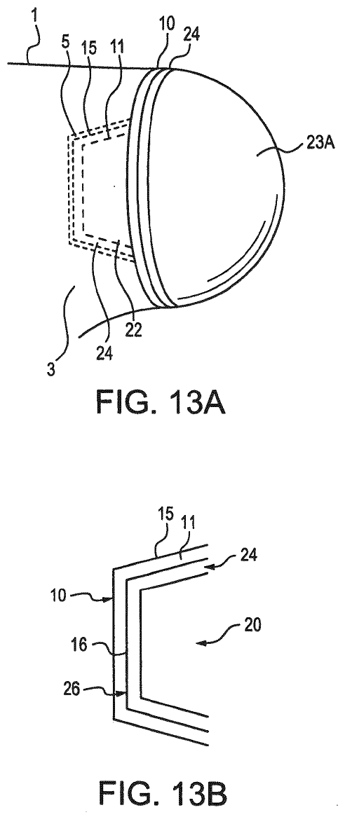

The articulating portion 20 of the novel glenoid implant 30 is then removably connected to the bone fixation portion 10 by means of the intermediate coupling portion 24 so as to allow later exchange of an articulating portion 20 having either a convex articulating surface 23A (FIGS. 12, 13A, 14) or a concave surface 23 (FIGS. 3, 4). In one embodiment, the connecting surfaces 16 of the bone fixation portion 10 and connecting surface 26 of the intermediate coupling portion 24 are of proper dimension and material to allow conical press fit connection of the articulating portion 20 to the bone fixation portion 10.

The articulating portion 20 of the novel glenoid implant 30 is then removably connected to the bone fixation portion 10 by means of the intermediate coupling portion 24 so as to allow later exchange of an articulating portion 20 having either a convex articulating surface 23A (FIGS. 12, 13A, 14) or a concave surface 23 (FIGS. 3, 4). In one embodiment, for example, the connecting surface 16 of the bone fixation portion 10 and connecting surface 26 of the intermediate coupling portion 24 are of proper dimension and material to allow a conical press fit connection of the portions 10 and 24. The press fit connection provides a durable but detachable connection or a stable, long-lasting but disengageable connection between the articulating portion 20 and the bone fixation portion 10. Other interactive surfaces may be substituted for the press fit connection so long as such substitutions provide one or more similarly durable but detachable connections. The connecting surface 22 of the articulating portion 20 and connecting surface 27 of the intermediate coupling portion 24 are of proper dimension and material to allow a durable interference fit connection of the portions 20 and 24. Additionally, the intermediate portion 24 may be of uniform material to the articulating portion 20 and provide the connecting surface for the articulating portion 20 as this connection is not required to be detachable.

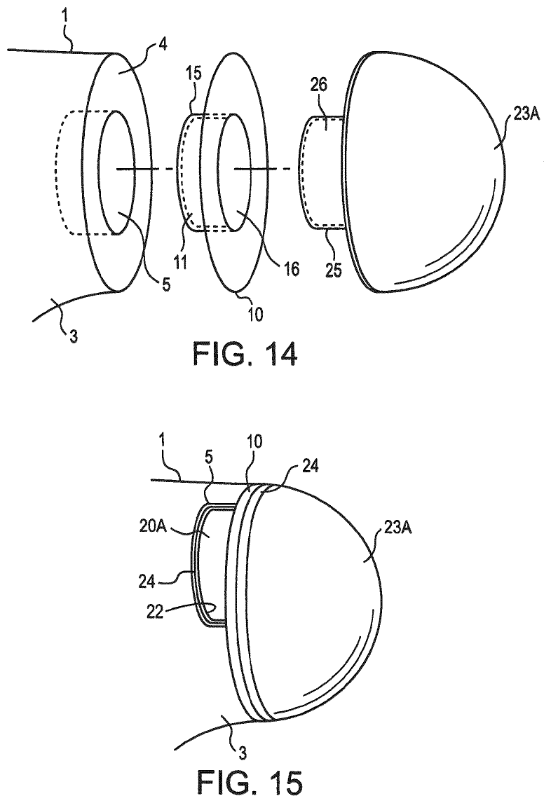

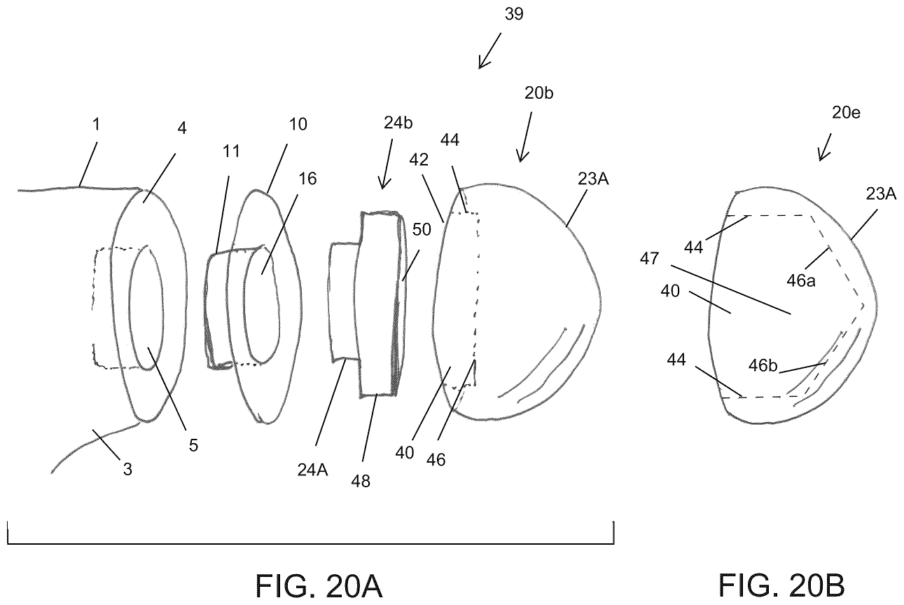

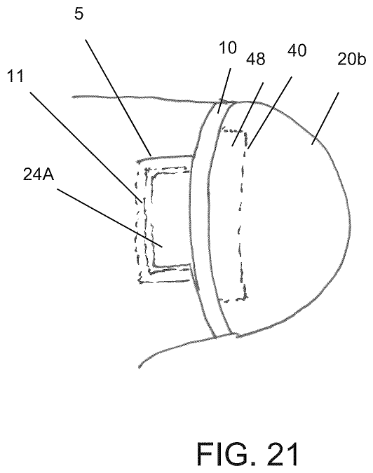

FIGS. 20A and 21 show another embodiment glenoid implant 39 comprising another embodiment intermediate coupling portion 24b is provided between the fixation portion 10 and another embodiment articulating portion 20b. The articulating portion 20b is the same as articulating portion 20 of FIG. 14 having a convex articulating portion, except articulating portion 20b lacks the protruding element 20A. Instead of a protruding element 20A, the articulating portion 20b comprises a recess 40 at a rear surface 42. In some embodiments, the recess is bounded by a side wall 44 and an end wall 46. The coupling portion 24b has a first portion 48 and the same protruding element 24A as coupling portion 24 has. The protruding element 24A extends from the coupling first portion 48. The first portion 48 is sized to fit within the recess 40 of the articulating portion. The first portion 48 is configured for durable long lasting but disengageable connection to the articulating portion with the recess 40. The first portion 48 and/or the sidewall 44 are sized and/or shaped to mate with each other such that the first portion 48 and the sidewall 44 form a durable but detachable connection or a stable, long-lasting but disengageable connection therebetween. In some embodiments, the first portion 48 has a width or diameter that is less than a width or diameter of the bone fixation portion 10 and/or the rim of the bone fixation portion 10. In some embodiments, the coupling portion 24b comprises a flat front side 50. The first portion 48 comprises a width or diameter that is greater than the width or diameter of the protruding element 24A. While the figures show that the first portion 48 and the side wall 44 are each cylindrical, other mating shapes are possible.

In some embodiments, another embodiment articulating portion 20e, shown in FIG. 20B can be used in place of articulating portion 20a Articulating portion 20e is identical to articulating portion 20a, except the recess 40 is enlarged to provide a hollow portion 47 extending adjacent articulating surface 23A. The end wall(s) 46a, 46b may be closer to the articulating surface 23A as compared to end wall 46 to provide the hollow portion 47 inside the articulating portion in front of the first portion 48 when the first portion is received 48 in the recess 40. While the 46a, 46b are shown as straight walls with an angular intersection between them, in some embodiments, the end wall is a curved continuous wall extending from the side wall 44.

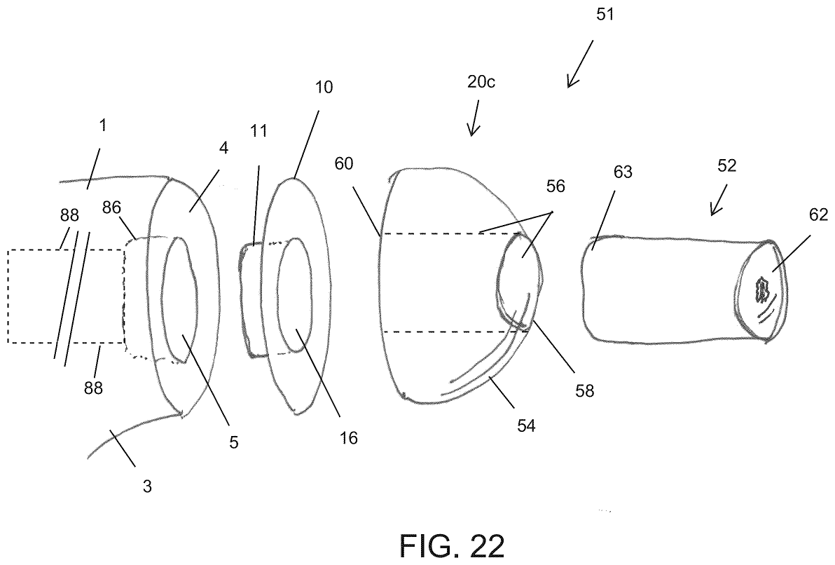

FIGS. 22, 23 and 24A show another embodiment glenoid implant 51 comprising the bone fixation portion 10, an articulating portion 20c, and a post 52. The articulating portion 20c is the same as the articulating portion 20 of FIG. 12, except without the protruding element 20A and with a post cannulation 56. The articulating portion 20c has a convex articulating surface 54 that is the same as the articulating surface 23A of FIG. 12 except, the articulating surface 54 is interrupted by a post cannulation 56. The cannulation extends from an aperture 58 in the front of the articulating surface through the articulating portion 20c to an aperture on the back side 60. In some embodiments, the aperture is at a center of the articulating surface.

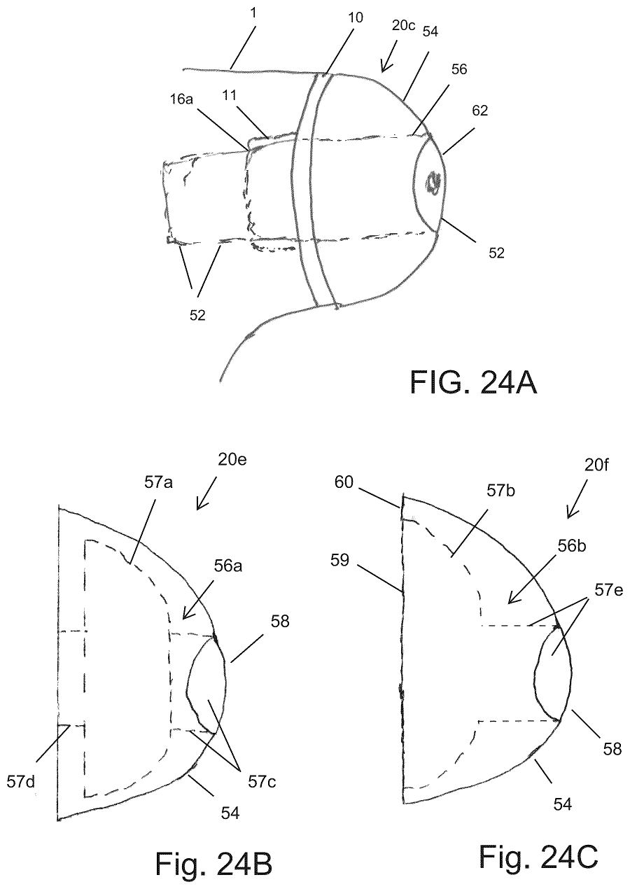

FIGS. 24B and 24C show alternative embodiment articulating portions 20e, 20f, which are the same as articulating portion 20c, except the post cannulation 56a, 56b comprises an enlarged portion 57a, 57b as compared to post cannulation 56. The enlarged portions 57a, 57b are enlarged from the remaining portions 57c, 57d, 57e. Therefore, the post cannulation can comprise a varying diameter or internal dimension along the length of the cannulation between aperture 58 in the front of the articulating surface 54 and the aperture 59 on the back side 60. Further, the aperture 59 can be enlarged as compared to aperture 58 as shown in FIG. 24C. The portion(s) 57c, 57d, 57e of the cannulation 56a, 56b can be sized so the wall(s) defining the cannulation friction fit to the post 52. The enlarged portions 57a, 57b create void in the articulating portion 20e, 20f that can reduce the weight of the articulating portion without compromising the stability of the connection between the articulating portion and the post. Articulating portions 20e, 20f can be used in place of articulating portion 20c.

The post 52 comprises a front 62. Preferably, the front 62 can be shaped to complete the convex articulating surface 54 at the aperture 58 when the post is seated in the post cannulation 56. Therefore, in some embodiments the combined post 52 and articulating portion 20c will substantially meet the same shape as articulating surface 23A of FIG. 12 when the post 52 is seated in the cannulation 56 as shown in FIG. 24A. In some embodiments, front 62 of the post 52 may optionally be flat or otherwise not comprising a completing convex shape, such as, without limitation, if the post has a small diameter or width.

In some embodiments, the glenoid cavity 5 may be prepared, such as reamed, to have a first portion 86 with a first width or diameter configured to receive the protruding element 11, and a second portion 88 with a second width or diameter configured to receive the post 52, such as shown in FIG. 22, 23, 24A. The first portion 86 has a larger width or diameter than the second portion 88.

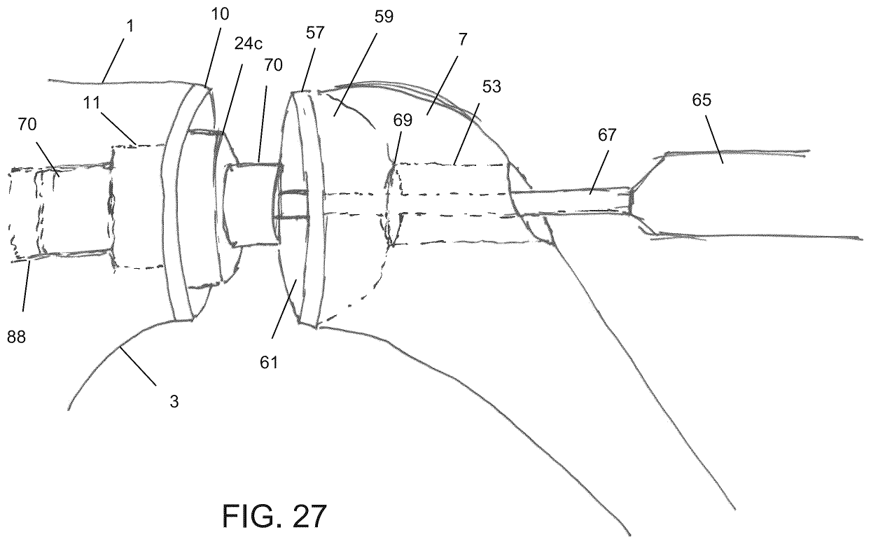

In FIG. 24A the post 52 is seated in the cannulation 56 of the articulating portion 20c. The post extends into the cannulation 16a of the bone fixation portion 10. In some embodiments, the post extends through the bone fixation portion and into the glenoid vault 5. The post may be releasably friction fitted within the cannulation of the articulating portion 20c and the bone fixation portion 10. In some embodiments, the post 52 comprises a conical threaded tip at penetrating end 63 of the post, such as the tip 78 of post 76 of FIG. 26 to engage the glenoid bone in or adjacent the cavity 5.

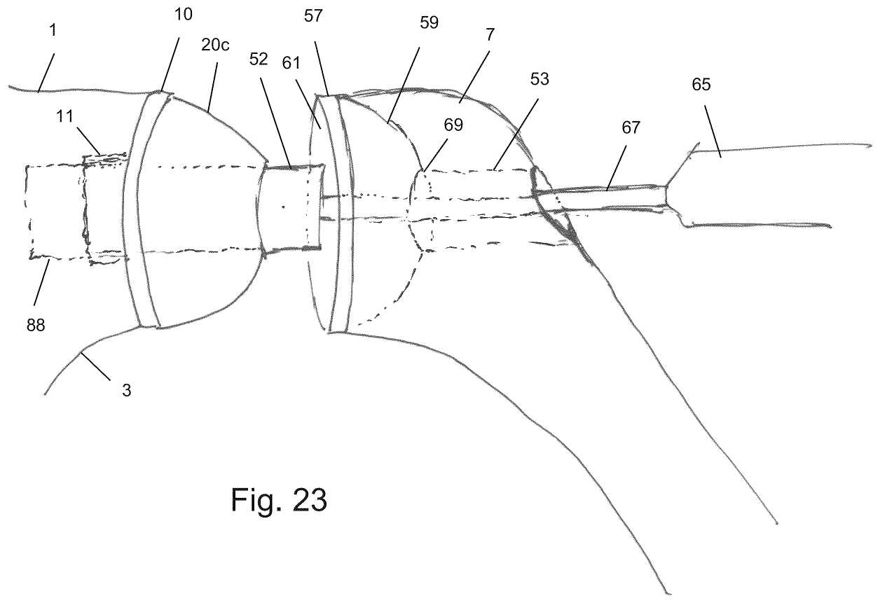

FIG. 23 shows one stage of implanting in a method of implanting the glenoid implant 51. The bone fixation portion 10 is fixed to the glenoid. The protruding element 11 is within the glenoid cavity 5. The articulating portion is placed on the bone fixation portion. The post 52 is inserted transhumerally through a transhumeral tunnel 53 with or without a humeral implant or portion 57, having a cannulation, of a humeral implant in place. And this can be performed while distracting the humerus from the glenoid without dislocating the shoulder joint. In some embodiments, the distracting is further performed without transversely cutting the rotator cuff.

An instrument 65, such as a driver, comprising a shaft 67 can be used to insert the post 52. The end of the shaft is placed against the front 62 of the post 52 or in some cases releasably fixed to the front 62. The post 52 can be driven by pushing the instrument, or hitting the instrument 65 with a hammer, or by rotating the instrument and rotating the attached post 52 to seat the post in the glenoid and the cannulation 56 of the articular portion 20c. When the portion 57 is in place, the portion 57 comprises a bone fixation side 59, an articular side 61 or a side for joining to an articular component, and a cannulation 69 extending from the bone fixation side to the articular side. The tunnel 53 and the cannulation 69 are sized to receive the post 52. In some embodiments, the humeral implant and the articular surface component are those described in U.S. patent application Ser. No. 18/211,396, filed Jun. 19, 2023 and that application is herein incorporated by reference.

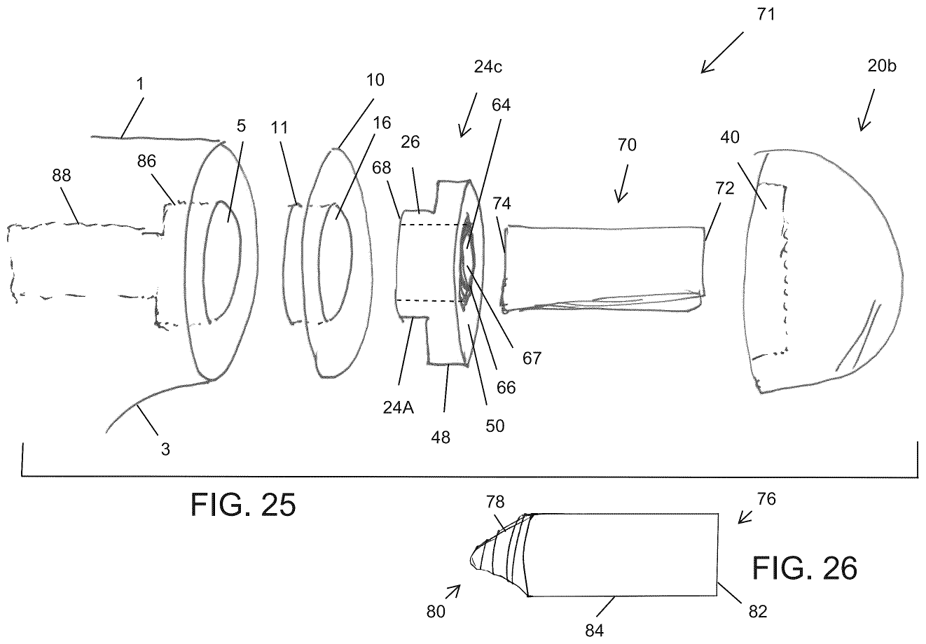

FIGS. 25, 27, and 28 show another embodiment glenoid implant 71 comprising the bone fixation portion 10, and the intermediate coupling portion 24c, an articulating portion 20b, and a post 70. The intermediate coupling portion 24c is identical to intermediate coupling portion 24b, except that coupling portion 24c comprises a cannulation 64. The cannulation 64 extends from an aperture 66 in the front side 50 through the coupling portion to an aperture in the back side 68. A connecting wall(s) or surface(s) 67 bounds and defines the cannulation in the coupling portion. In some embodiments, the post 70 comprises a uniform cross-section along its length from a first end 72 to a second end 74. In some embodiments, a second embodiment post 76 is used in place of post 70. Post 76 comprises a threaded conical tip 78 at a second end 80 opposite the first end 82. In some embodiments, the post 76 comprises a main portion 84 comprising a uniform cross-section along its length from the first end 82 to the conical tip 78.

In some embodiments, the glenoid cavity 5 may be prepared, such as reamed, to have a first portion 86 with a first width or diameter configured to receive the protruding element 11, and a second portion 88 with a second width or diameter configured to receive the post 70, 76 as shown in FIG. 25. The first portion 86 has a larger width or diameter than the second portion 88.

FIG. 27 shows one stage of implantation or one method of implanting the glenoid implant 71. The bone fixation portion 10 is fixed to the glenoid. The protruding element 11 is within the glenoid cavity 5 and portion 86. The protruding element 24A of coupling portion 24c is engaged with the connecting surface 16 and the cannulation 16a of the bone fixation portion 10. The post 70 is inserted in the cannulation 64 and is long lastingly fixed in the glenoid. The post 72 is inserted transhumerally through a transhumeral tunnel 53 with or without a humeral implant or portion 57, having a cannulation, of a humeral implant in place.

The instrument 65 comprising the shaft 67 can be used to insert the post 70, 76. The end of the shaft is placed against the front 72, 82 of the post 70, 76 or in some cases releasably fixed to the front 72, 82. The post 70, 76 can be driven by pushing the instrument, or hitting the instrument 65 with a hammer, or by rotating the instrument and rotating the attached post 70, 72 to seat the post in the glenoid and the cannulation 64 of the coupling portion 24c. The tunnel 53 and the cannulation 64 are sized to receive the post 70, 76.

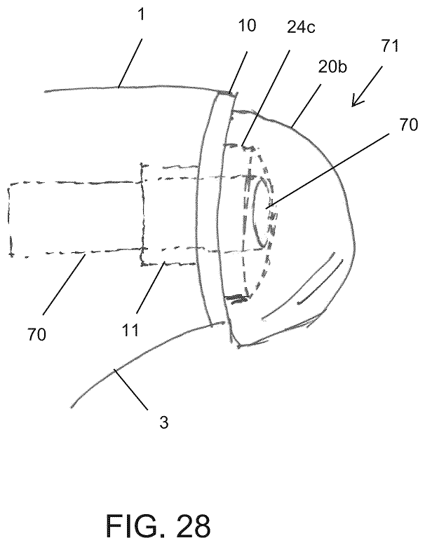

FIG. 28 shows the implant 71 implanted in the glenoid. The front end 72 of the post 70 can be flush with, recessed behind, or protruded from the front 50 of the coupling portion 24c. A portion of the post 70 and the coupling portion 24c are seated in the recess 40 of the articulating portion 20b. In some embodiments, the articulating portion 20b is vertically or otherwise offset from a center of the bone fixation portion and from a center of the glenoid cavity 5. While the articulating portion 20b is shown in FIG. 28 with a vertically inferior offset, the offset could be in any direction. The protruding element 11 of the bone fixation portion is seated in the cavity 5 and in portion 86, the second end 74 of the post 70 is in the glenoid cavity and in portion 88. The post 70 extends through the cannulation 16a in the bone fixation portion 10.

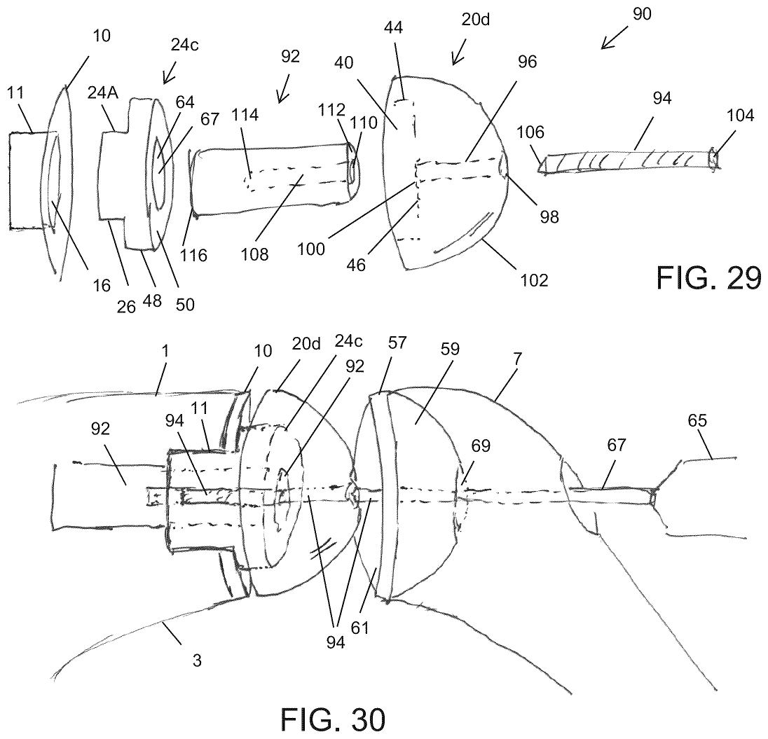

FIG. 29 shows another embodiment glenoid implant 90 comprising the bone fixation portion 10, and intermediate coupling portion 24c, an articulating portion 20d, a first post 92, and a second post 94. The articular portion 20d is identical to articulating portion 20b, except articular portion 20d comprises a cannulation 96. The cannulation 96 extends from an aperture 98 on the front convex articulating surface 102 to an aperture 100 in the end wall 46 of the recess 40.

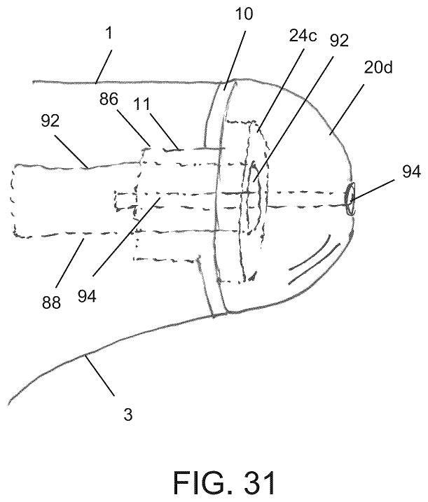

The second post 94 comprises a first end 104 and a second end 106. The second end 106 may comprise a conical and or threaded tip. The first end 104 may comprise a flat surface or a surface shaped to complete the convex articulating surface 102 at the aperture 98 when the post 94 is seated in the cannulation 96. Therefore, in some embodiments the combined post 94 and articulating portion 20d will substantially meet the same shape as articulating surface 23A of FIG. 12 when the post 94 is seated in the cannulation 96 as shown in FIG. 31.

The first post 92 comprises a cannulation 108. The cannulation 108 extends from an aperture 110 on the first end 112 to a blind terminal end 114 within the post 92. The first end 112 is opposite of the second penetrating end 116. In some embodiments, the first post 92 has a conical threaded tip at the second end, the same as post 76.

When the implant 90 is implanted, the protruding element 11 of the bone fixation portion is in portion 86 of the cavity 5; protruding element 24A is received in cannulation 16a and engaged with connecting surface 16; post 92 is engaged with cannulation 64 and received and engaged in portion 88 of the cavity 5, the front end 112 of the post 92 may be flush with front side 50; first portion 48 of coupling portion 24c is engaged with the recess 40 of articulating portion 20d; the second post 94 is engaged with the cannulation 108 of the post 92 and cannulation 96 of the articulating portion 20d; the end 106 of the post 94 may be at, adjacent or engaged with the end 114 of the cannulation 108 of the post 92; and, the first end 104 of the second post 94 is flush with the articulating surface 102 at the aperture 98, as shown in FIG. 31.

FIG. 30 shows a stage of implanting of one method of implanting the glenoid implant 90. The bone fixation portion 10 is fixed to the glenoid. The protruding element 11 is within the glenoid cavity 5 and portion 86. The protruding element 24A of coupling portion 24c is engaged with the connecting surface 16 and the cannulation 16a of the bone fixation portion 10. The post 92 is inserted in the cannulation 64 and durably and long lastingly fixed therein but disconnectable. The post 92 is inserted transhumerally through a transhumeral tunnel 53 with or without a humeral implant or portion 57, having a cannulation, of a humeral implant in place, as described regarding implant 71 and post 70. The post 94 is inserted transhumerally through a transhumeral tunnel 53 with or without a humeral implant or portion 57, having a cannulation, of a humeral implant in place and is seated in the first post 92 at the cannulation 108 and the articulating portion 20d at the cannulation 96.

The instrument 65 comprising the shaft 67 can be used to insert the second post 94. The end of the shaft is placed against the front 104 of the post 94 or in some cases releasably fixed to the front 104. The post 94 can be driven by pushing the instrument, or hitting the instrument 65 with a hammer, or by rotating the instrument and rotating the attached post 94 to seat the post in the cannulation 96 of the articulating portion 20d and in the cannulation 108 of the second post 92. The tunnel 53 and the cannulations 96, 108 are sized to receive the posts 94. The tunnel 53 and the cannulation 64 are sized to receive the post 92.

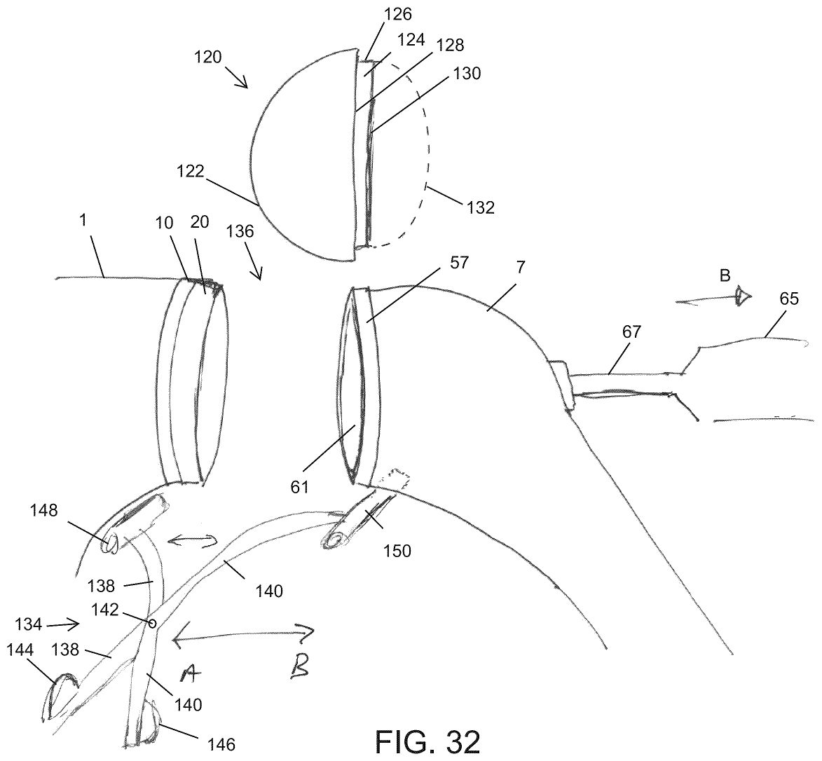

A trial humeral articular component 120 is shown in FIG. 32. The word trial as used in connection with components indicates components that are not intended to stay within the body of a human patient after the medical procedure, such as surgery, is complete. Trial components are distinguished from implant components. Implant components are components that are intended to stay within the body of a human patient after the medical procedure, such as surgery, is complete.

The trial component 120 has an articular surface 122 and a coupling portion 124. The coupling portion is for connecting to a bone fixation portion 57 of a humeral implant, such as disclosed in U.S. patent application Ser. No. 18/211,396, filed Jun. 19, 2023. The coupling portion 124 comprises a low-profile depth 126 between the end 128 of the articular surface and the terminal end 130 of the coupling portion. The depth 126 of the coupling portion 124 is low-profile compared to that of the coupling portion of an implant articular component 132 shown in broken lines in FIG. 32. In some embodiments, the low-profile depth 126 of the coupling portion 124 is in the range of 0.25 millimeter (mm) to 5 mm, inclusive.

A method of determining the appropriate size of a humeral implant or glenoid implant or articulating components thereof is disclosed. The method comprises insertion and removal in situ of one or more trial articular component(s) 120, 154, 170, 180, 181, 194, 210, 220, 230, 250 in the shoulder joint without dislocating the shoulder joint. In some embodiments of the method, the insertion and/or removal is with distraction and without dislocating the shoulder joint, and with or without transversely cutting the rotator cuff.

In some embodiments of the method, the method comprises inserting and removing one or more trial articular components 154, 180, 181, 181, 194, 210, 220, 250 in the shoulder joint at the glenoid without dislocating the shoulder joint. In some embodiments, the method comprises inserting and removing one or more trial articular components 120, 170, 194, 210, 220, 230 in the shoulder joint at the humeral head without dislocating the shoulder joint. Before removing the trial articular component(s), the joint is tested for proper fit and movement, such as by visual inspection and/or moving the humerus. The humerus may be moved through all or a portion of an anatomic range of motion of the humerus or the humeral head of the proximal humerus. The trial articular component is then removed.

If testing that trial articular component shows that the component was the proper size or otherwise fit, then the surgeon can proceed to insert/place an implant humeral articulating component or an implant glenoid articulating component that corresponds in size and/or otherwise to the trial articular component. If the trial articular component did not meet the surgeon's satisfaction as to size, fit, operation or otherwise, a different trial articular component, having a different size or other characteristic can be place or fixed according to the above method to attempt to find the proper sized, shaped, or otherwise fit implant articular component by temporarily placing or fixing one or more other different trial humeral or glenoid articular components, then testing, then removing the same. The trial articular component comprises the low-profile coupling portion for easing insertion and removal without dislocating the shoulder joint, and with or without transversely cutting the rotator cuff.

In some embodiments of the method, before or after the trial articular component is placed, the glenoid implant, such as any of the glenoid implants having a concave articular surface, such as surface 23, disclosed herein is placed/inserted in the glenoid 1. And, a bone fixation portion 57 of a humeral implant is placed or fixed to a prepared portion of the proximal humerus 7, such as shown in FIG. 32. The humeral implant and/or glenoid implant, and/or components thereof may be placed or fixed at the respective humerus or glenoid without dislocating the shoulder joint, with or without transversely cutting the rotator cuff. The components, such as articulating components of an implant may be inserted and placed or fixed at or on other components of the implant at the humerus and/or glenoid without dislocating the shoulder joint, with or without transversely cutting the rotator cuff. Distraction may be applied to distract the humerus away from the glenoid without dislocating the shoulder joint when the humeral implant and/or glenoid implant, and/or components thereof may be inserted and placed or fixed at the respective humerus or glenoid.

In some embodiments of the method, distraction is used before and/or during when the trial articular component is inserted. The distraction can be achieved by one or more means, such as manually pulling the humeral head away from the glenoid; using an instrument, such as instrument 65, to pull the humeral head away from the glenoid; using a distractor, such as distractors 134, 290 to push the humeral head away from the glenoid; and/or using the trial articular component to distract such as by driving the trial articular component between the glenoid and the humeral head thereby causing the humeral head to distract from the glenoid without dislocation.

In some embodiments of the method, when a distractor 134 is used to distract or separate in the directions A, B the proximal humerus and the glenoid from each other, and more particularly the articulating surface of the glenoid implant from the implanted portions of the humeral implant, such as the bone fixation portion 57 without dislocating the shoulder joint with or without transversely cutting the rotator cuff. An instrument 65 can also be used to, with or without the distractor 134, apply distraction forces on the proximal humerus by grabbing and pulling in the direction B away from the glenoid. The distractor creates a gap 136 sufficient to receive the trial humeral articular component 120 therein. The distractor 134 can also be used to distract or separate in the directions A, B the proximal humerus and the glenoid from each other before the glenoid implant is implanted and/or before the bone fixation portion 57 of a humeral implant is placed or fixed to a prepared portion of the proximal humerus 7.

Referring to FIG. 32, in some embodiments of the method, the trial humeral articular component 120 is inserted at and mounted to the bone fixation portion 57 at the articular side 61 via the gap 136. Distraction when used is released. If the distractor is used, the distractor is released or reversed to allow trial humeral articular component 120 to contact the articulating portion 20 of the glenoid implant. The instrument 65, if used, is released or moved toward the glenoid. If distraction is achieved by driving the trial articular component between the glenoid and humeral head, distraction will be released by the trial articular components mounting to the bone fixation portion and the coupling portion 124 being received at the articular side 61 of the bone fixation portion 57.

Then the joint is tested for proper fit and movement, such as by visual inspection and/or moving the humerus. The humerus may be moved through all or a portion of an anatomic range of motion of the humerus or the humeral head of the proximal humerus. The trial humeral articular component 120 is then removed by using the distractor 134 to separate the proximal humerus from the glenoid without dislocating the shoulder joint, and optionally without transversely cutting the rotator cuff. Then the trial humeral articular component 120 is removed.

If testing that trial humeral articular component 120 shows that the component was the proper size or otherwise fit, then the surgeon can proceed to insert/place an implant humeral articular component that corresponds in size and/or otherwise to the trial humeral articular component 120. If the trial humeral articular component 120 did not meet the surgeon's satisfaction as to size, fit, operation or otherwise, a different trial humeral articular component, having a different size or other characteristic can be place or fixed according to the above method to attempt to find the proper sized, shaped, or otherwise fit implant humeral articular component by temporarily placing or fixing one or more other different trial humeral articular components, then testing, then removing the same, which each trial humeral articular component comprises the low-profile coupling portion 124 for easing insertion and removal without dislocating the shoulder joint, and with or without transversely cutting the rotator cuff. The low-profile coupling portion 124 requires the width of the gap 136 to be less than would otherwise be required with a full depth 132 of a coupling portion of an implant articular component.

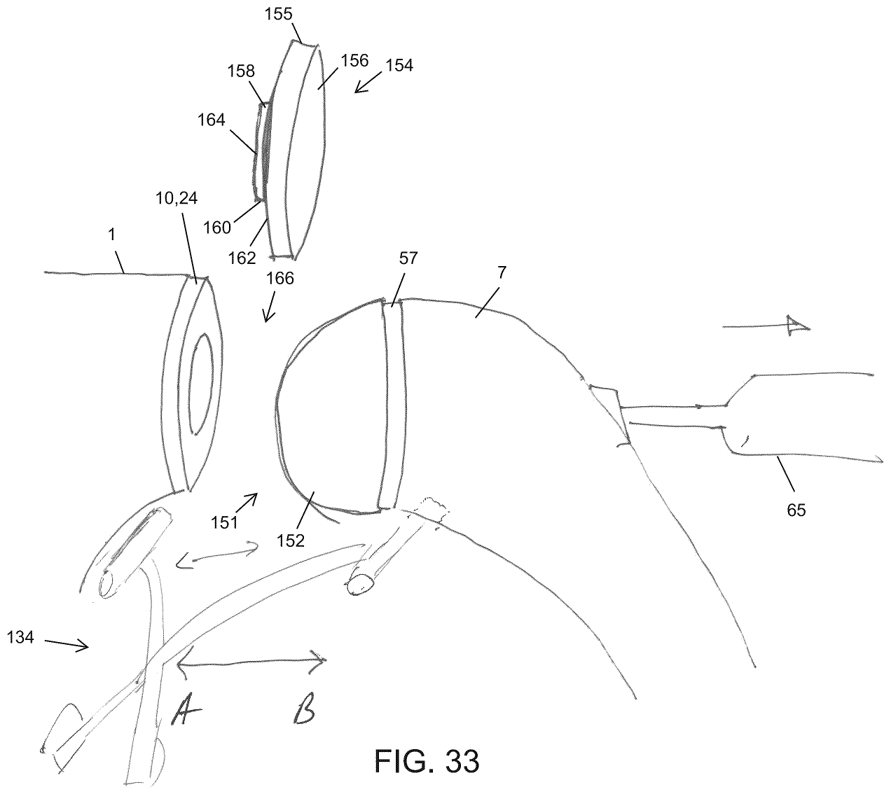

In some embodiments, distractor 134 comprises a first arm 138 and a second arm 140. The arms 138, 140 are pivotally connected to each other at an overlap location which may comprise a pivot pin 142 extending through, and pivotally joining, each of the arms. The arms are curved or in a v-shape so that they overlap but curve away from each other along at least some segment(s) moving away from the overlap and pin 142 as shown in FIG. 32. The arms 138, 140 each comprise a grasping loop 144, 146 at a first end and engaging members or bars 148, 150 at an opposite end as shown in FIG. 33. When the grasping loops are drawn together the engaging bars 148, 150 move apart in directions A and B. When grasping loops are drawn apart the engaging bars 148, 150 move together. In some embodiments, the arms 138, 140 are configured to cross at the overlap location so that when the grasping loops are drawn together the engaging members or bars 148, 150 move together and when grasping loops are drawn apart the engaging members or bars 148, 150 move part in directions A and B.

FIG. 33 shows a trial articulating component 154 of a glenoid implant. The articulating component 154 comprises a first portion 155 and a protruding element 158. The first portion 155 comprises an articulating surface 156. The articulating surface 156 may be the same or similar to articulating surface 23. The protruding element 158 comprises a low-profile depth 160 between the end 162 of the first portion 155 and the terminal end 164 of the protruding element 158. The protruding element 158 is low-profile compared to the depth of the protruding element 20A of articulating portion 20. In some embodiments, the low-profile depth 160 of the protruding element 158 is in the range of 0.25 millimeter (mm) to 5 mm, inclusive.

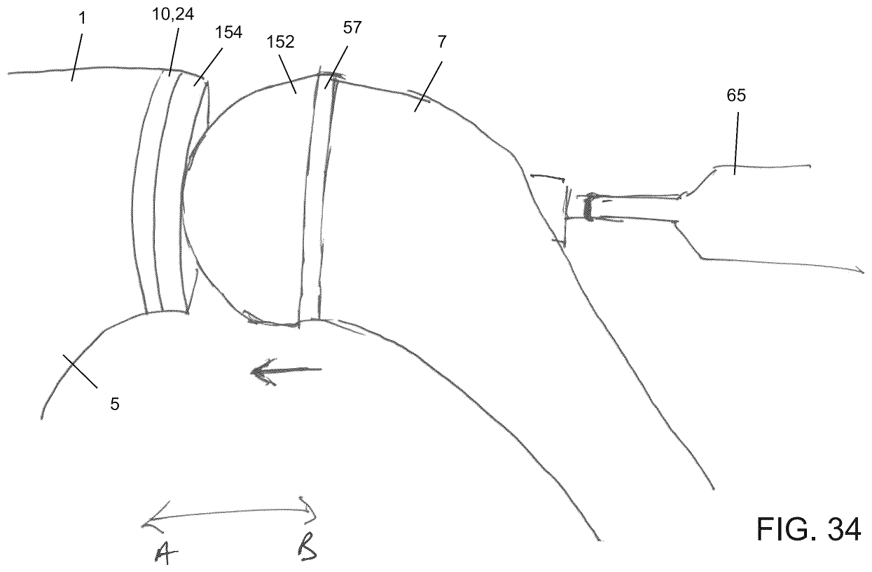

Another embodiment of the method of determining the appropriate size of a humeral implant or glenoid implant or articulating components thereof is disclosed and explained in relation to FIGS. 33 and 34. The method comprises insertion and removal in situ of a trial articular component(s) 154 in the shoulder joint without dislocating the shoulder joint. In some embodiments of the method, the insertion and/or removal is with distraction and without dislocating the shoulder joint, and with or without transversely cutting the rotator cuff.

In some embodiments of the method, before or after the trial articular component is placed, a humeral implant 151, is placed/inserted on a prepared portion of the proximal humerus. The implant may comprise a bone fixation portion 57 and an articular portion 152. In some embodiments, the implant may comprise the humeral implant of in U.S. patent application Ser. No. 18/211,396, filed Jun. 19, 2023. The bone fixation portion 10 of the glenoid implant is placed or implanted at the glenoid vault 5. In the case where an intermediate coupling portion 24 is used, the intermediate coupling portion 24 is placed or fixed to the bone fixation component. The distractor 134 can also be used to distract or separate in the directions A, B the proximal humerus and the glenoid from each other before placing and/or fixing the bone fixation portion 57, the articular portion 152, and/or the bone fixation portion 10. The humeral implant and/or glenoid implant, and/or components thereof may be placed or fixed at the respective humerus or glenoid without dislocating the shoulder joint, with or without transversely cutting the rotator cuff. The components, such as articulating components of an implant may be inserted and placed or fixed at or on other components of the implant at the humerus and/or glenoid without dislocating the shoulder joint, with or without transversely cutting the rotator cuff. Distraction may be applied to distract the humerus away from the glenoid without dislocating the shoulder joint when the humeral implant and/or glenoid implant, and/or components thereof may be inserted and placed or fixed at the respective humerus or glenoid.

In some embodiments of the method, when a distractor 134 is used, the distractor 134 is used to distract or separate in the directions A, B the proximal humerus and the glenoid from each other, and more particularly the articulating portion 152 of the humeral implant from the implanted portions 10, 24 of the glenoid implant without dislocating the shoulder joint and optionally without transversely cutting the rotator cuff. The distractor creates a gap 166 sufficient to receive the trial articulating component 154 therein. The instrument 65 can also be used to distract the proximal humerus by grabbing and/or pulling in the direction B away from the glenoid.

The trial articulating component 154 is mounted to the bone fixation portion 10, or to the coupling portion 24 if used, via the gap 166. Distraction, when used is released. The distractor 134 is released or reversed to allow the articular portion 152 of the humeral implant to contact the trial articulating component 154 as shown in FIG. 34. The instrument 65, if used, is released or moved toward the glenoid. If distraction is achieved by driving the trial articular component between the glenoid and humeral head, distraction will be released by the trial articular components mounting to the bone fixation portion 10, or to the coupling portion 24 if used and the coupling portion 158 being received within bone fixation portion 10, or to the coupling portion 24 if used.

Then the joint is tested for proper fit and movement, such as by visual inspection and/or moving the humerus. The humerus may be moved through all or a portion of an anatomic range of motion of the humerus or the humeral head of the proximal humerus. The trial articulating component 154 is then removed by using the distractor 134 to separate the proximal humerus from the glenoid without dislocating the shoulder joint and optionally without transversely cutting the rotator cuff. Then trial humeral articular component 154 is removed. If testing that trial articulating component 154 shows that the component 154 was the proper size or otherwise fit, then the surgeon can proceed to insert an implant articulating component, such as portion 20, that corresponds in size and/or otherwise to the trial articulating component 154. If the trial articulating component 154 did not meet the surgeon's satisfaction as to size, fit, operation or otherwise, a different trial articulating component, having a different size or other characteristic can be placed or fixed according to the above method to attempt to find the proper sized, shaped, or otherwise fit implant articulating component by temporarily placing or fixing one or more other different trial articulating components, then testing, then removing the same, where each trial articulating component comprises the low-profile protruding element 158 for ease of insertion and removal without dislocating the shoulder joint and with or without transversely cutting the rotator cuff. The low-profile protruding element 158 requires the depth of the gap 166 to be less than would otherwise be required with a full depth of the protruding element 20A of an implant articular component 20.

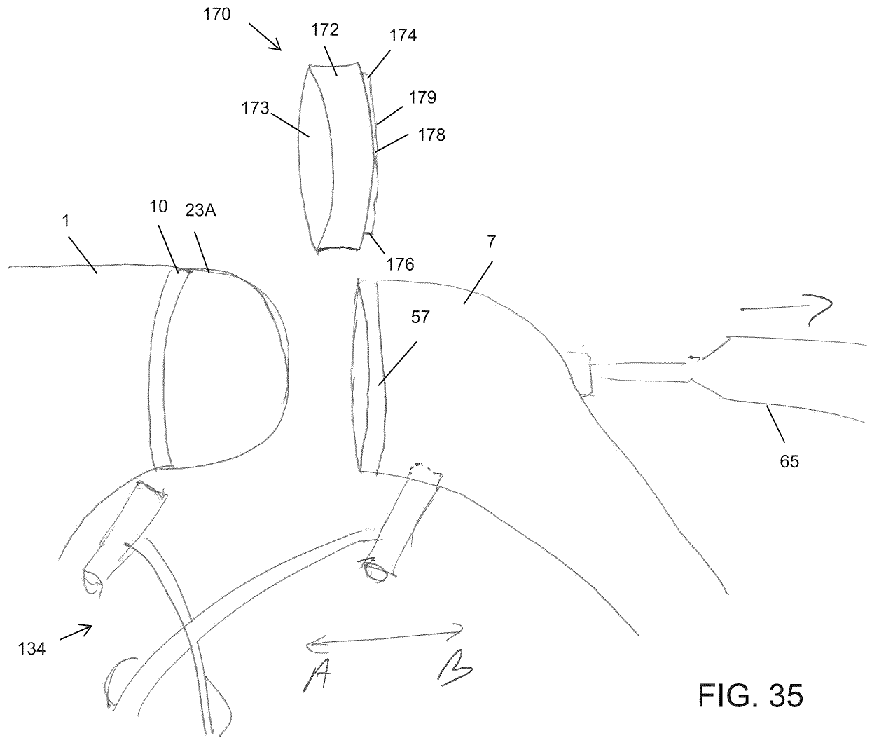

Another embodiment trial humeral articulating component 170 is shown in FIG. 35. The component 170 has an articular portion 172 and a coupling portion 174. The articulating portion 172 has a concave articular surface 173, which, in some embodiments, has a shape that is the same or similar to articulating surface identified at number 25 in U.S. patent application Ser. No. 18/211,396, filed Jun. 19, 2023. The coupling portion 174 is for connecting to a bone fixation portion 57 of a humeral implant. The coupling portion 174 comprises a low-profile depth 176 between the end 178 of the articular portion and the terminal end 179 of the coupling portion. The trial coupling portion 174 is low-profile compared to the depth of the implant coupling portion 132, such as shown in broken lines in FIG. 32, of an implant articular component. The trial humeral articulating component 170 can be used in the method of determining the appropriate size of a humeral implant or glenoid implant or implant articulating components thereof described herein regarding trial humeral articular component 120.

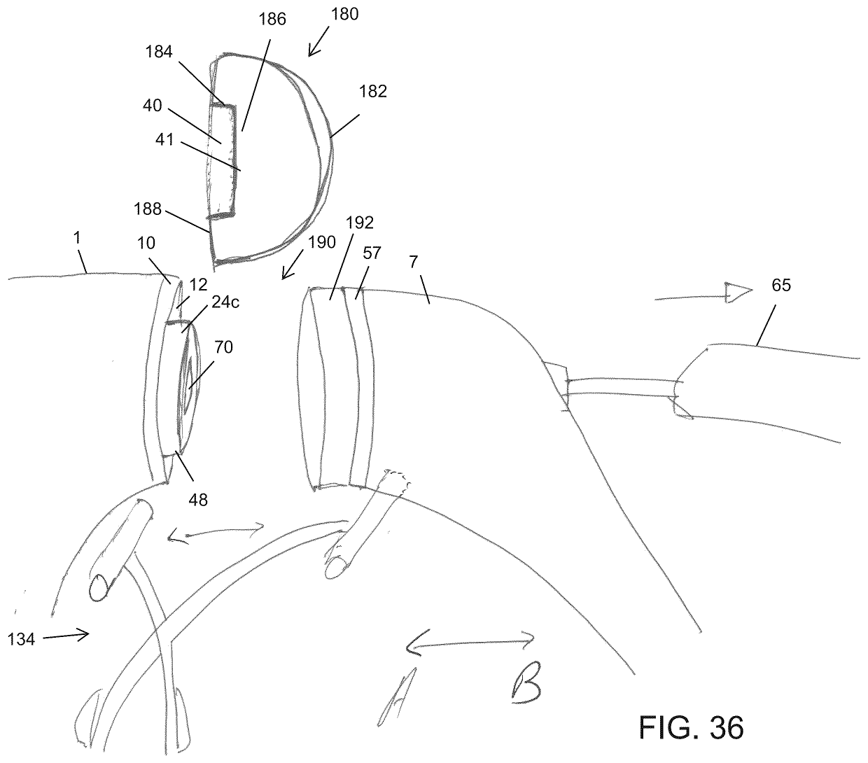

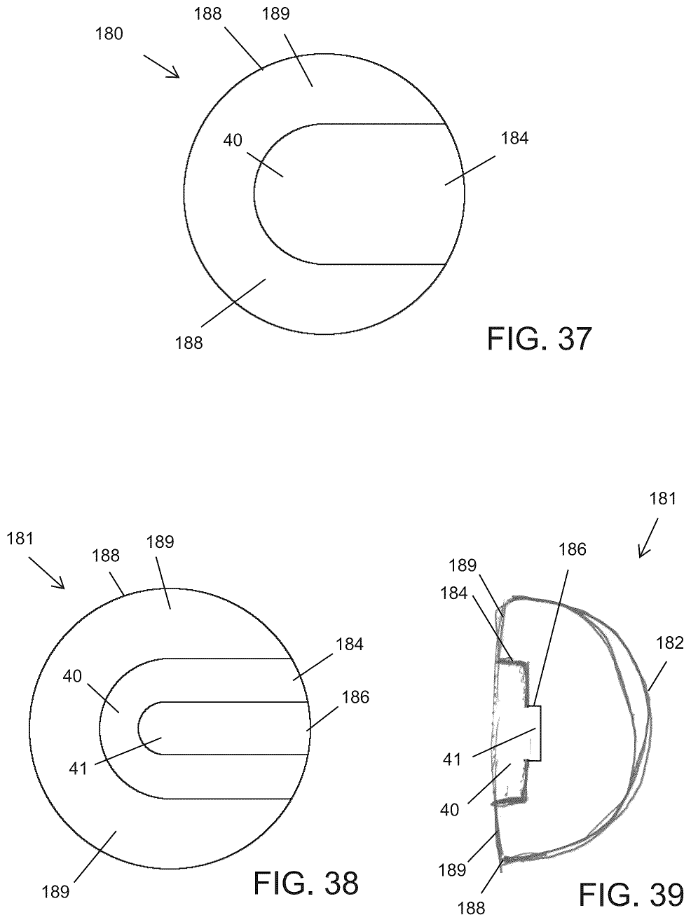

Another embodiment trial glenoid articular components 180, 181 are shown in FIGS. 36 to 39. The articular component 180 comprises an articular surface 182. The articular component 180 has the same or similar shape to articulating portion 20b, except that the articular surface has a first side channel 184 in communication with and providing access to the recess 40. The first side aperture of the first side channel 184 is at or adjacent to a back edge 188 of the articular surface 182 and back side 189 of the articular component 180. The first side channel is open on a side of the articular component providing an opening in the articular surface adjacent the back edge 188 as shown in FIG. 36.

Trial glenoid articular component 181 is the same as trial glenoid articular component 180 except that trial articular component 181 comprises a second recess 41 concentric with the first recess 40, and a second side channel 186 in communication with and providing access to the second recess 41. The second recess 41 is configured to receive a front portion of the post 70, when the post is not mounted flush with the front 50 of coupling portion 24c. The second side channel 186 is at or adjacent to a back edge 188 of the articular surface 182 and back side 189 of the articular component 181. When post 70 is mounted flush with the front 50 of the coupling portion 24c trial glenoid articular component 180 is used. The second side channel 186 is open on a side of the articular component 181 providing an opening in the articular surface 182 adjacent the back edge 188 as shown in FIG. 39. The second side channel 186 extends deeper into the articular component 181 and the articular surface 182 than the first side channel 184 as shown in FIG. 39.

The first side channel 184 allows the articular component 180 to slide laterally onto the coupling portion 24c while the back edge 188 or side of the articular component 180 maintains contact with, or is closely proximate to, the front of the rim 12 of the bone fixation portion 10, or the glenoid such as if the bone fixation portion 10 is rimless. The coupling portion 24c will enter the trial articular component 180 via the channel 184 and travels along the channel to the recess 40 as the trial articular component moves onto the coupling portion 24c. This allows the articular component 180 to be placed on the coupling portion 24c with a smaller gap 190 between the glenoid and the articular component 192 of a humeral implant and a smaller gap between the glenoid and the humerus 7 as compared to the implant articular surface portion 20b that has no side channel in communication with the recess 40. When post 70 is positioned protruded from the front 50 of the coupling portion 24c, the post 70 enters the trial articular component 181 via the channel 186 and travels along the channel to the second recess 41 as the trial articular component 181 is moved onto the coupling portion 24c.

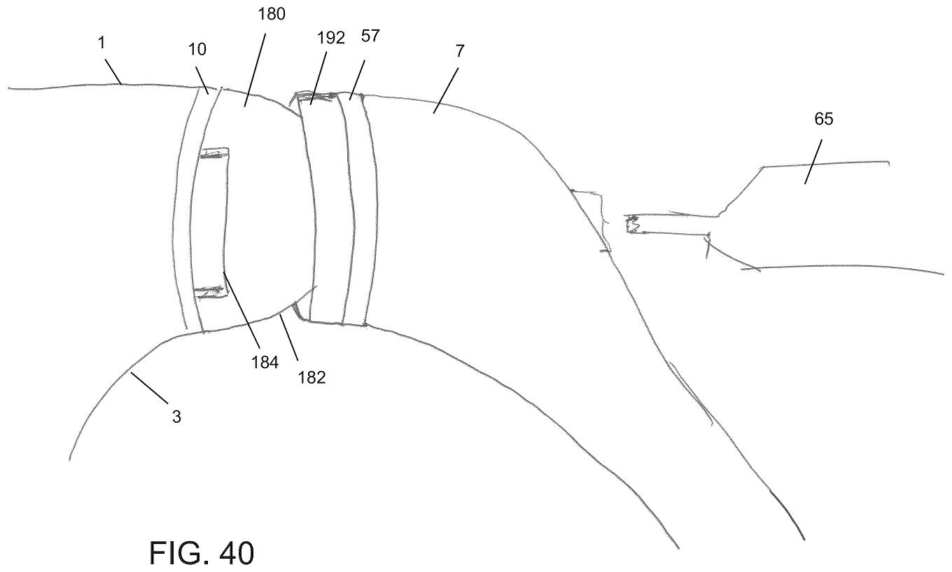

The trial articular component 180, 181 can be used in the method(s) of determining the appropriate size of a humeral implant or glenoid implant or articulating components thereof described herein regarding trial articular component 154. When the trial articular component 180 is placed on the coupling portion 24c, articular component 180, 181 is slid laterally onto the coupling portion 24c through the first side channel 184 while the back edge 188 or side of the articular component 180 maintains contact with, or is closely proximate to, the front of the rim 12 of the bone fixation portion 10, or the glenoid such as if the bone fixation portion 10 is rimless. When the trial articular component 181 is placed on the coupling portion 24c, articular component 181 is slid laterally onto the coupling portion 24c through the first side channel 184, and the post 70 is received through the second side channel 186, while the back edge 188 or side of the articular component 181 maintains contact with or is closely proximate to, the front of the rim 12 of the bone fixation portion 10, or the glenoid such as if the bone fixation portion 10 is rimless. FIG. 40 shows trial articular component 180 deployed in a join and ready for visual and/or movement testing and inspection.

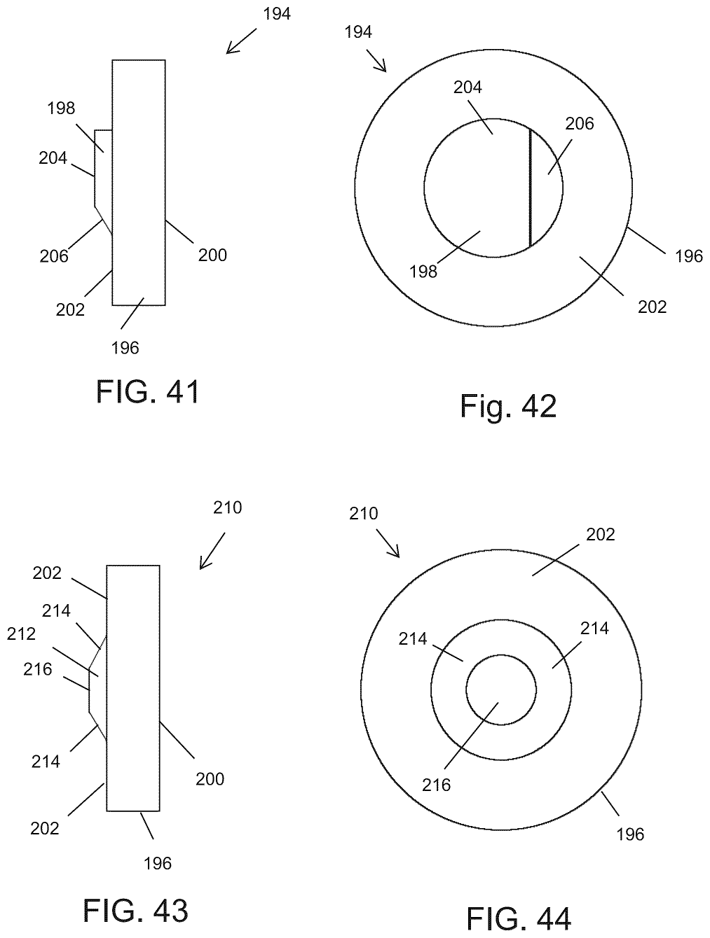

FIGS. 41 and 42 show another embodiment trial articulating component 194 for a glenoid implant or humeral implant and usable with the glenoid implants and/or humeral implants disclosed herein. Trial articulating component 194 may comprise a different depth, diameter/dimension, and/or articulating surface curvature depending on whether it is configured to be deployed at the glenoid or at the humerus. The component 194 comprises an articular portion 196 and a protruding element 198. The articular portion comprises an articulating surface 200, which in some embodiments is the same as articulating surface 23. The protruding element comprises a plateau 204 and a ramp 206. The ramp 206 extends from a back 202 of the articular portion 196 to plateau 204 providing a transition therebetween. In some embodiments, the protruding element 198 comprises a low-profile depth as described with respect to protruding element 158. The ramp provides additional ease of insertion/placement and/removal of the articular component 194. The ramp may be slid or driven over the rim 12 of the bone fixation portion 10 or the coupling portion 24 when the component 194 is placed or removed from the bone fixation portion 10 or the coupling portion 24, such as during the method(s) of determining the appropriate size of a humeral implant or glenoid implant or articulating thereof components described herein. The ramp may be slid or driven over component 57 of a humeral implant when the component 194 is placed or removed from the bone fixation component 57, such as during the method of determining the appropriate size of a humeral implant or glenoid implant or articulating components thereof described herein.

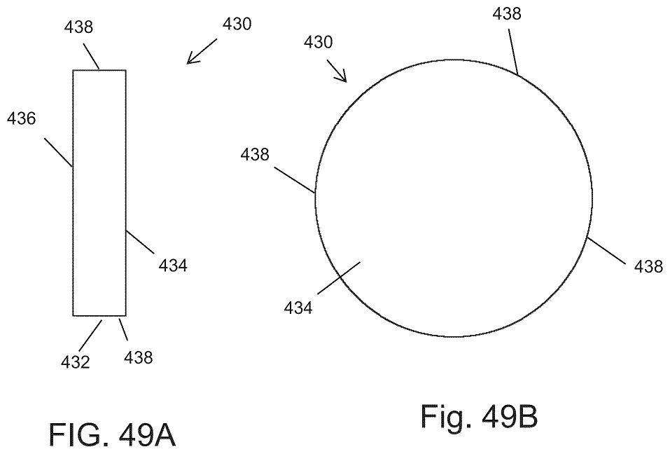

A measuring block 430 is disclosed for a glenoid implant or humeral implant and usable with the glenoid implants and humeral implants disclosed herein. An exemplary measuring block is shown in FIGS. 49A, 49B. The measuring block has a predefined thickness 432 between a first side 434 and a second side 436. The measuring block may have a uniform thickness throughout its body or a portion thereof. When the measuring block is placed within the shoulder joint, the block can indicate the distance between the glenoid or a component thereof and the humeral head or a component thereof. While the block is shown as a disk or cylinder, the block can be other shapes such as quadrilateral, square, or rectangle with or without rounded corners. One or more perimeter edges 438 may comprise a ramp (not shown) similar to ramp 206. A method of determining the appropriate size of a humeral implant or glenoid implant or articulating components thereof is disclosed. One or more measuring blocks having a known thickness(es) can be placed in the shoulder joint to measure the distance between the glenoid or a component thereof and the humeral head or a component thereof. When a measuring block having a known thickness is placed in the shoulder joint and opposite sides 434, 436 of the block touch the glenoid or a component thereof and the humeral head or a component thereof, then that thickness is a measure of the distance between the glenoid or a component thereof and the humeral head or a component thereof. Then the surgeon can proceed to insert/place an implant humeral articulating component and/or an implant glenoid articulating component that corresponds in size and/or otherwise to the measurement achieved via the measuring block. This measuring block can be used without dislocating the shoulder joint or without transversely cutting the rotator cuff.

FIGS. 43 and 44 show another embodiment trial articular component 210 for a glenoid implant or humeral implant and usable with the glenoid implants and humeral implants disclosed herein. The component 210 comprises an articular portion 196 and a protruding element 212. The component 210 is the same as component 194 except the ramp 241 of the protruding element 212 surrounds the plateau 216 instead of extending from one side.

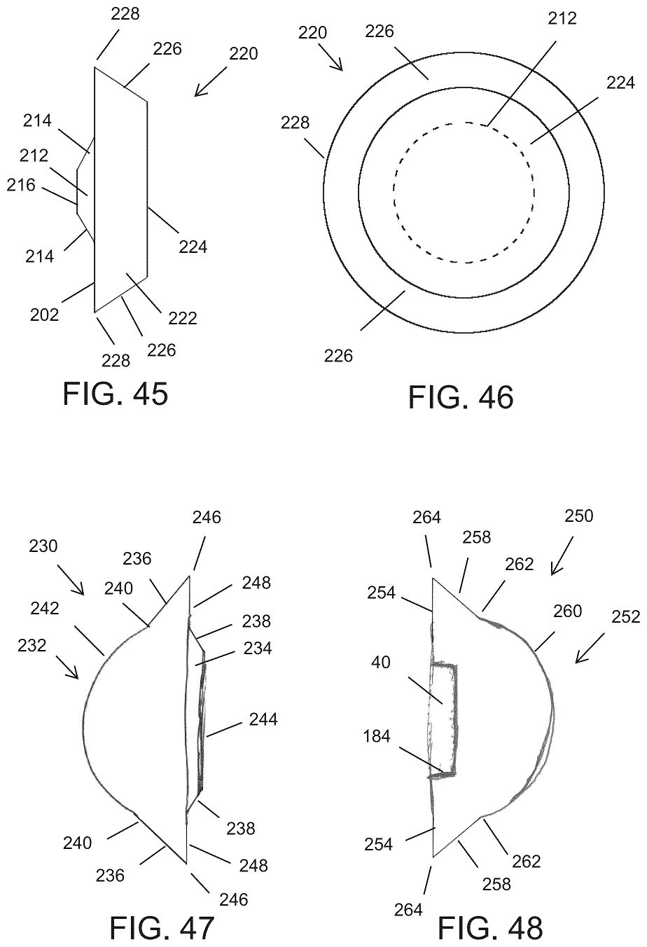

FIGS. 45 and 46 show another embodiment trial articular component 220 for a glenoid implant or humeral implant and usable with the glenoid implants and humeral implants disclosed herein. The component 220 comprises an articular portion 222 and a protruding element 212. The component 220 is the same as component 210 except the articular portion 222 comprises a second ramp 226. The ramp 226 extends from an edge 228 of the articular portion 222 to a main portion 224 comprising the articular surface providing a transition therebetween. The ramp 226 may form a wedge at the edge 228 between the ramp 226 and a back 202 of the articular portion. The second ramp encircles the main portion 224 and extends about the perimeter of the articular portion 222. The second ramp 226 provides additional ease of insertion/placement and/removal of the articular component 220. The second ramp may be slid or driven in front of the articular portion 152 of the humeral implant when the component 220 is placed or removed from the bone fixation portion 10 or the coupling portion 24, such as during the method(s) of determining the appropriate size of a humeral implant or glenoid implant or articulating components thereof described herein. The second ramp may be slid or driven in front of the articular portion 23A of the glenoid implant when the component 220 is placed or removed from the bone fixation portion 57, such as during the method(s) of determining the appropriate size of a humeral implant or glenoid implant or articulating components described herein.

The ramp 226 and or ramp 214 may be used to drive the bone fixation portion 10 or the coupling portion 24 from the articular portion 152 of the humeral implant, and therefore to drive the humerus 7 away from the glenoid 1 to generate a larger gap 166 and/or to assist in distracting the humerus from the glenoid without dislocating the shoulder joint with or without transversely cutting the rotator cuff, while inserting the trial articular component 220. FIG. 47 shows another embodiment trial humeral articular component 230 for a humeral implant. The component 230 comprises an articular portion 232 and a coupling portion 234. The component 230 is the same as component 120 except the articular portion 232 comprises a perimeter first ramp 236 and the coupling portion comprises a second ramp 238. The ramp 236 surrounds the main articular surface 242 and merges 240 with the main articular surface 242 about its perimeter. The coupling portion 234 comprises a second ramp 238 surrounding a plateau 244, which is the same or similar to protruding element 212. The ramp 236 may form a wedge at an edge 246 between the ramp 236 and a back 248 of the articular portion.

The ramp 236 provides additional ease of insertion/placement and/removal of the articular component 230. The ramp 236 may be slid or driven in front of the articulating portion 20 of the glenoid implant when the component 230 is placed or removed from the bone fixation portion 57, such as during the method of determining the appropriate size of a humeral implant or glenoid implant or articulating thereof components described herein. The ramp 236 and or ramp 238 may be used to drive the bone fixation portion 57 of the humeral implant away from the articulating portion 20 of the glenoid implant, and therefore to drive the humerus 7 away from the glenoid 1 to generate a larger gap 136 and/or to assist in distracting the humerus from the glenoid without dislocating the shoulder joint and with or without transversely cutting the rotator cuff, while inserting the trial articular component 230.

FIG. 48 shows another embodiment trial articular component 250 for a glenoid implant. The component 250 comprises an articular portion 252 and a back side 254. The component 250 is the same as component 180 except the articular portion 252 comprises a perimeter ramp 258. The ramp 258 surrounds the main articular surface 260 and merges 262 with the main articular surface 260 about its perimeter. The ramp 258 may form a wedge at an edge 264 between the ramp 258 and a back 254 of the articular portion. The articular portion 252 has a first side channel 184 in communication with and providing access to the recess 40. In some embodiments, the opening to the first side channel 184 interrupts the ramp 258 at or adjacent the back 254.

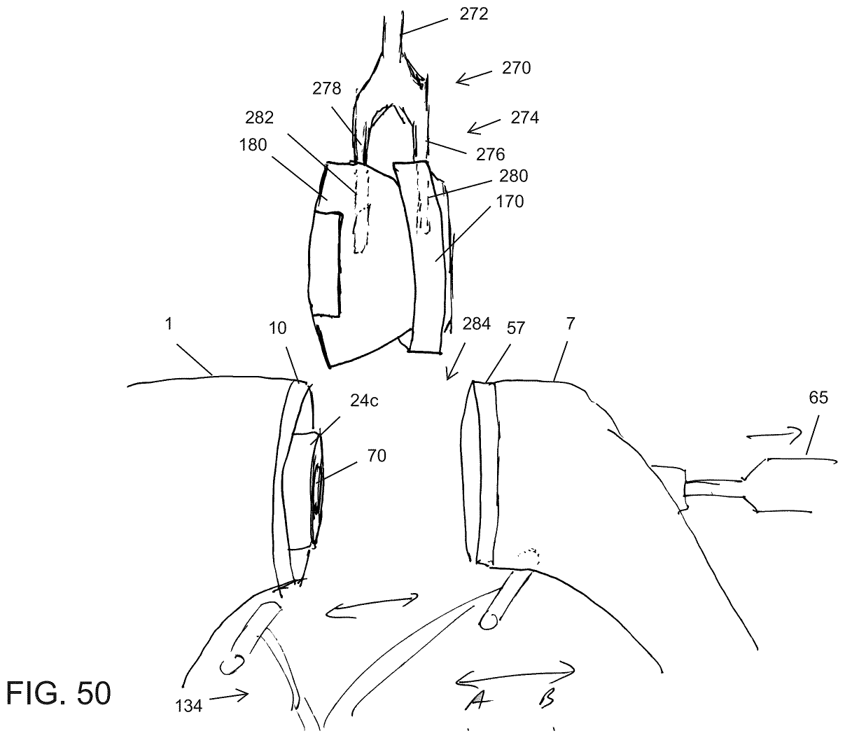

The ramp 258 provides additional ease of insertion/placement and/removal of the articular component 250. The ramp 258 may be slid or driven in front of the articular component 192 of the humeral implant when the component 250 is placed or removed from the coupling portion 24c, such as during the method(s) of determining the appropriate size of a humeral implant or glenoid implant or articulating components thereof described herein. The ramp may be used to drive the articular component 192 of the humeral implant away from bone fixation portion 10 and the coupling portion 24c, and therefore to drive the humerus 7 away from the glenoid 1 to generate a larger gap 190 and/or to assist in distracting the humerus from the glenoid without dislocating the shoulder joint and with or without transversely cutting the rotator cuff, while inserting the trial articular component 250. FIG. 50 shows a fork insertion instrument 270 comprising a shaft 272 and a fork portion 274. The shaft 272 may comprise a handle (not shown). The fork portion comprises a plurality of spaced apart prongs 276, 278. A first prong 276 connects to a first implant component, such as trial articulating component 170. A second prong 278 connects to a second implant component, such as trial articular component 180. The instrument 270 holds the articular components, such as components 170, 180 in a mated relationship during insertion, as shown in FIG. 50, for simultaneous insertion and/or removal. The components may each comprise an instrument channel 280, 282 for receiving a corresponding prong 276, 278 of the instrument 270. The channels 280, 282 may be sized to frictionally receive the prongs 276, 278 so that the prongs frictionally hold the articular components 170, 180 until a predefined removal force is applied to separate the prongs from the articular components.

Another method of determining the appropriate size of a humeral implant or glenoid implant or articulating components thereof is disclosed. The method comprises insertion and removal in situ in the shoulder joint without dislocating the shoulder joint. In some embodiments of the method, the insertion and/or removal is with distraction and without dislocating the shoulder joint, and with or without transversely cutting the rotator cuff. The humerus 7 is drawn back from the glenoid, such as with the distractor 134 and/or other instruments, such as instrument 65. In some embodiments, the bone fixation portions 10, 57 of the humeral implant and the glenoid implant are placed or fixed in the corresponding prepared glenoid vault 5, and the prepared portion of the proximal humerus 7. The coupling portion 24c is placed or fixed to the bone fixation portion 10 of the glenoid implant. Post 70 is inserted through the transhumeral tunnel (not shown in FIG. 50) and is seated in the coupling portion 24c and the glenoid. The trial articulating components 170, 180 are mated together with the articular surfaces of each in contact with the other. The prongs 276, 278 are inserted into the channels 280, 282 of the corresponding components 170, 180 and are frictionally held thereto. The instrument 270 is moved toward the gap 284 between the glenoid and the humerus. The mated articular components 170, 180 are moved via the instrument 270 into the gap 284. Articulating component 170 may be aligned with and mounted to bone fixation portion 57 of the humeral implant at the same or substantially the same time as the articular component 180 is mounted to the coupling portion 24c of the glenoid implant. The instrument 270 is withdrawn from each of the components 170, 180, which may be achieved by holding the components in place with a second instrument and withdrawing the instrument 270 to overcome the friction force holding the prongs 276, 278 in the channels 280, 282. In some embodiments, the components 170, 180 are mounted to portion 57, coupling portion 24c, such that the mounting provides sufficient holding force to withdraw the instrument 270 from the components 170, 180 overcoming the holding friction of the prongs 276, 278 in the channels 280, 282.

The distractor 134 is released or reversed to allow the articular portion 152 of the humeral implant and the trial articulating component 154 to be loaded against each other, as shown in FIG. 34. The instrument 65, if used, is released or moved toward the glenoid to allow the humerus to move toward the glenoid. In some embodiments, the instrument 270 is withdrawn from the components 170, 180 after the distractor is released and or the instrument 65 is released or moved toward the glenoid.

Then the joint is tested for proper fit and movement, such as by visual inspection and/or moving the humerus so that the humeral articulating component articulates against the glenoid articulating surface/component. The humerus may be moved through all or a portion of an anatomic range of motion of the humerus or the humeral head of the proximal humerus.

One or both of the trial articular components 170, 180 are then removed by using the distractor 134 to separate the proximal humerus from the glenoid without dislocating the shoulder joint with or without transversely cutting the rotator cuff. The instrument 270 can be reinserted into the components 170, 180 and then the components 170, 180 are jointly withdrawn from the joint.

In the alternative, only one of the components 170, 180 may be removed from the joint, while the other remains for further testing with another trial articular component to be next inserted.

If testing the trial articular components 170, 180 shows that the components were the proper size or otherwise fit, then the surgeon can proceed to place/insert an implant articulating component, such as portion 20, 192, that corresponds in size and/or otherwise to the corresponding trial articulating component 170, 180. If the trial articulating component 170, 180 did not meet the surgeon's satisfaction as to size, fit, operation or otherwise, a different trial articulating component, having a different size or other characteristic can be placed or fixed according to the above method to attempt to find the proper sized, shaped, or otherwise fit implant articulating component by temporarily placing or fixing one or more other different trial articulating components, then testing, then removing the same. While, the instrument is shown with trial articular components 170, 180 it can be used with other mateable glenoid/humeral trial articular components 120, 154, 181, 194, 210, 220, 230, 250 disclosed herein. In some embodiments, glenoid and humeral trial articular components are a unitary component rather than two separate components. In some embodiments, the instrument 270 is formed together with the glenoid and humeral trial articular components as a unitary component rather than separate components.

In some embodiments, the instrument 270 may be used with humeral implants and/or glenoid implants or components thereof, such as a humeral articulating component and/or a glenoid articulating component. The instrument 270 may engage and hold a humeral articulating component and/or a glenoid articulating component in the same way as the instrument 270 is described above as holding trial components 170, 180. The implant articular components can be mated, inserted, aligned, and mounted to bone fixation portion 57 of the humeral implant and the coupling portion 24c of the glenoid implant in the same manner as described for trial components 170, 180. The mated implant articular components are moved via the instrument 270 into the gap 284. The humeral articulating component may be aligned with and mounted to bone fixation portion 57 of the humeral implant at the same or substantially the same time as the glenoid articular component is mounted to the coupling portion 24c of the glenoid implant. The instrument 270 is withdrawn from each of the implant components, which may be achieved by holding the implant components in place with a second instrument and withdrawing the instrument 270 to overcome the friction force holding the prongs 276, 278 in the instrument channels. One or both of the mated components can also be removed, such as described above for trial components 170, 180. In some embodiments, the instrument 270 may be configured to grip an exterior of the implant articulating component or trial articulating component rather than or in addition to being received in instrument channels.

In some embodiments, the instrument 270 may be used with a trial articulating component on a first side and an implant articulating component on the other side. A trial articular component is mated to an implant articular component. The instrument 270 may engage and hold the implant articulating component mated together with the trial articulating component in the same way as the instrument 270 is described above as holding trial components 170, 180. The mated trial and implant articular components can be mated, inserted, aligned, and mounted to bone fixation portion 57 of the humeral implant and the coupling portion 24c of the glenoid implant in the same manner as described for trial components 170, 180. One or both of the mated components can also be removed, such as described above for trial components 170, 180.

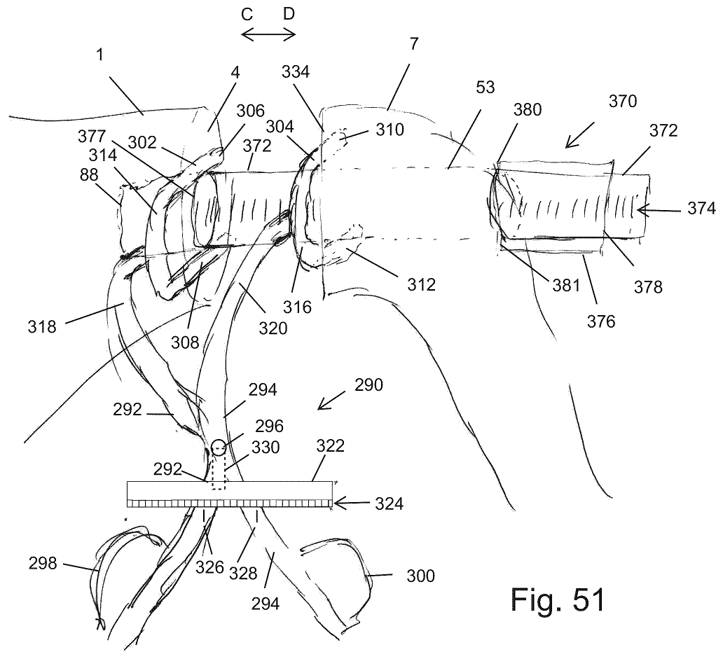

Another embodiment distractor 290 is shown in FIG. 51. The distractor 290 comprises a first arm 292 and a second arm 294. The arms 292, 294 are pivotally connected to each other at an overlap location which may comprise a pivot pin 296 extending through, and pivotally joining, each of the arms. The arms 292, 294 each comprise a grasping loop 298, 300 at a first end and forks 302, 304 at a second end. Each fork comprises two prongs 306, 308, 310, 312 joined by a cross member 314, 316. In some embodiments, the prongs are formed together with the cross member as a unitary component. Ends 318, 320 of the arms 292, 294 are bent so that the forks are transverse to the arms below the bends, such as at the pin 296. In some embodiments, the arms 292, 294 are bent so that the forks are perpendicular to the arms below the bends, such as at the pin 296. Therefore, the loops 298, 300 are offset from the forks 302, 304. When the grasping loops are drawn together the forks 302, 304 move apart in directions C and D. When grasping loops are drawn apart the forks 302, 304 move together. In some embodiments, the arms 292, 294 are configured to cross at the overlap location so that when the grasping loops are drawn together the forks 302, 304 move together and when grasping loops are drawn apart the forks 302, 304 move part in directions C and D.

The distractor 290 comprises a distraction indicator 322. In some embodiments, the distraction indicator comprises a plurality of measurement indicators 324. The measurement indicators may comprise markings, grooves, ridges, or other visual indications provided along the distraction indicator 322. The indicators may be arranged according to a scale of distance measurement, such as millimeters, centimeters, or inches (including fractions of an inch). One or both of the arms 292, 294 comprise reading indicators 326, 328, which may comprise markings, grooves, ridges, or other visual indications.

In some embodiments, the distraction indicator is connected to and supported by the pin 296, via a support arm 330. The support arm is fixed to the pin and to the distraction indicator. The arms 292, 294 are movable relative to the distraction indicator. When the arms move the associated reading indicators also move and therefore move along the distraction indicator. The distractor is calibrated so that the distance between the forks is indicated on the distraction indicator between the reading indicators 326, 328. Therefore, a user can read the distance between the forks on the distraction indicator 322.

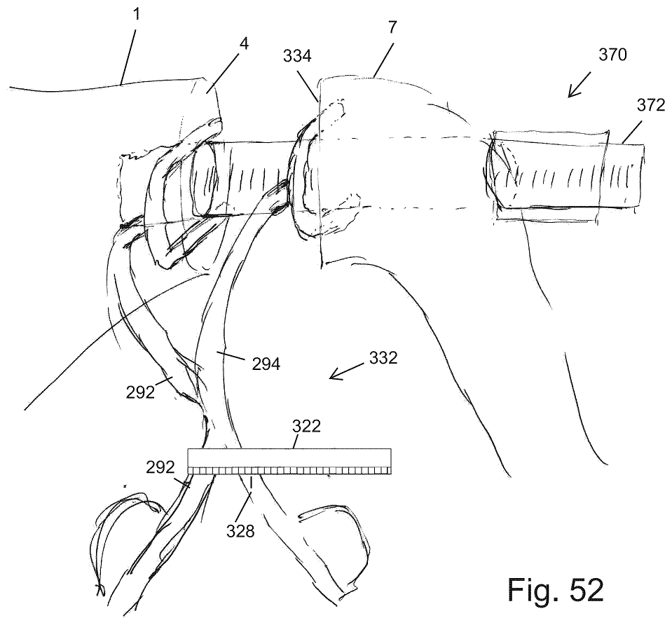

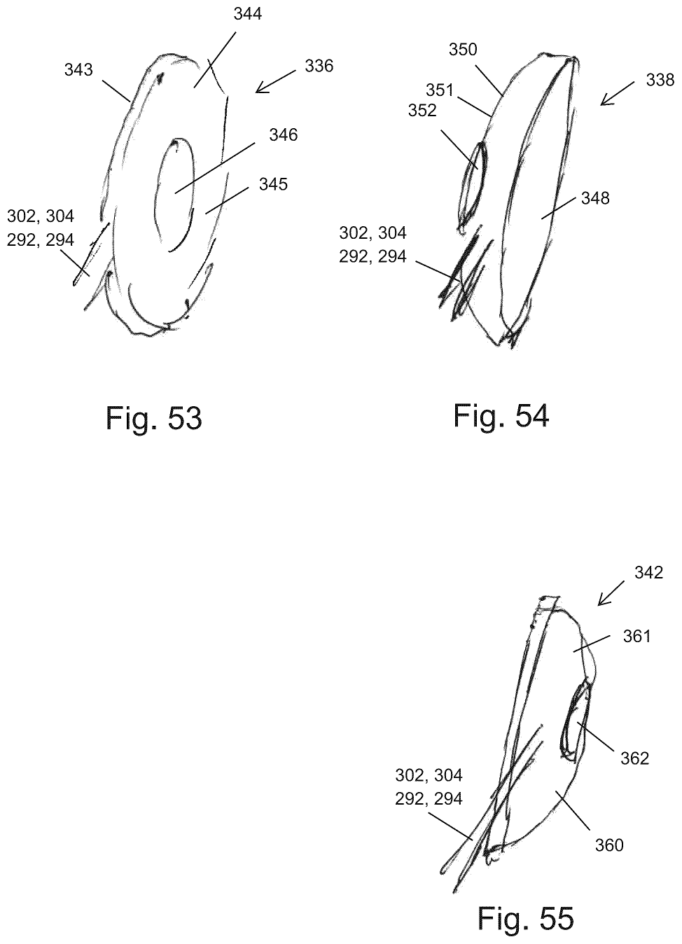

In some embodiments, the distraction indicator is fixed to one of arms 292 or 294 and the other of arms 292 or 294 comprises the reading indicator 326 or 328. FIG. 52 shows another embodiment distractor 332, which is the same as distractor 290 except that the distraction indicator is fixed to arm 292 and the reading indicator 328 is on arm 294. Therefore, the movement of the arm that the distraction indicator is fixed to will move the distraction indicator relative to the reading indicator. The movement of the arm with the reading indicator will move the reading indicator relative to the distraction indicator. The distraction indicator is calibrated so that the distance between the forks is indicated on the distraction indicator at the reading indicator. Therefore, the distractor 290, 332 can be applied between the glenoid and the humerus to measure the distance between as shown in the distraction indicator 322. In particular the distractor forks 302, 304 can be applied to (1) a native glenoid surface, the prepared glenoid surface 4, or the bone fixation component 10 on one side and (2) the native humeral head surface, the prepared humeral surface 334, or the bone fixation component 57 on the other side to measure the distance between as shown in the distraction indicator 322. Further, the forks can be applied against placed or install components of glenoid and/or humeral implants, such as the bone fixation portion 10, 57 as described and/or articulating portion 20, 20b, 20c, 20d, 20e, 120, 152, 192 and/or trial components 120, 154, 170, 180, 181, 194, 210, 220, 230, 250.

In some embodiments, the distractor is used with one or more paddles 336, 338, 342. The paddles can be deployed on one or both forks 302, 304, or in some embodiments, can be attached to the end of the arms 292, 294 in place of one or both the forks 302, 304.

Each of the paddles comprises a body 344, 348, 360. The paddles may comprise a through-going cannulation 346, 352, 362. The body is connected to one of the forks 302, 304 or arms 292, 294. The paddle 336 comprises a flat or substantially flat or concave front surface 345 can be deployed against prepared glenoid bone surface 4 or the bone fixation portion 10 of the glenoid implant on one side and/or the prepared humeral surface 334 or the bone fixation portion 57 of the humeral implant. The paddle may have a flat back surface 343.

The paddles 338, 342 with a convex surface 351, 361 can be deployed against the bone fixation portions 10, 57 of the glenoid and/or humeral implant. In some embodiments, the paddle 338 is configured to engage the bone fixation portion 10 of the glenoid implant. Paddle 342 is configured to engaged the bone fixation portion 57 of the humeral implant or a prepared surface of the humeral head.

Therefore, the paddles can be selected for each side of the distractor based on whether the glenoid and/or humerus have prepared surfaces 4, 334 exposed or whether bone fixation components 10, 57 for the glenoid and/or the humerus are deployed or implanted.

A measuring device 370 is shown in FIGS. 51 and 52. The device 370 comprises an elongated shaft 372. The shaft has a plurality of measurement indicators 374 spaced apart along a length of the shaft 372. The measurement indicators 374 may comprise markings, grooves, ridges, or other visual indications provided along the shaft. The indicators may be arranged according to a scale of distance measurement, such as millimeters, centimeters, or inches (including fractions of an inch). The shaft is placed through the transhumeral tunnel 53. The shaft extends into the shoulder joint between the glenoid and the humerus. In some applications, the shaft 372 will stop at the prepared surface 4 and not extend into the glenoid cavity 5. In some embodiments, the distance between the prepared surfaces 4, 334 can be determined by reading the measurement indicators 374 between surfaces 4, 334 of the glenoid and the humerus. In some applications, the shaft 372 will be placed and extend into the glenoid cavity 5.

In some embodiments, the shaft 372 is placed through the transhumeral tunnel 53 and into the glenoid cavity 5 to maintain the glenoid and humerus in proper alignment for making measurements, including when the soft tissue bridging the shoulder joint is tensioned, such as when the joint is under distraction. Further the shaft 372 can be placed through bone fixation components 10, 57, and/or other implant components, between the humerus and the glenoid cavity 5 when aligning.

In some embodiments, the distraction measuring device 370 is a depth gauge. The device comprises a sleeve. The depth that the shaft is deployed at relative to a home position of the shaft is shown by the measurement indicator 374 aligned at the end 378 of the sleeve. The home position of the shaft is where the front 377 of the shaft is aligned with the end 381 of the sleeve 376. The distance between opposing surfaces of the shoulder joint, such as (1) a native glenoid surface, the prepared glenoid surface 4, or the bone fixation component 10 on one side and (2) a native humeral head surface, the prepared humeral surface 334, or the bone fixation component 57 on the other side can be determined by reading the measurement indicator 374 at the end 378 of the sleeve. In some cases, the depth of the humerus between the opening 380 to the transhumeral tunnel 53 and a native humeral head surface, the prepared humeral surface 334 or the bone fixation component is subtracted from the measurement indicator shown at the end 378 of the sleeve to determine the distance between opposing surfaces of the shoulder joint, such as (1) a native glenoid surface, the prepared glenoid surface 4, or the bone fixation component 10 on one side and (2) a native humeral head surface, the prepared humeral surface 334, or the bone fixation component 57 on the other side.

Another method of determining the appropriate size of a humeral implant or glenoid implant or articulating components thereof is disclosed. The method comprises using a distractor 290, 332 to measure the distance between opposing surfaces of the shoulder joint, such as (1) a native glenoid surface, the prepared glenoid surface 4, or the bone fixation component 10 and (2) a native humeral head surface, the prepared humeral surface 334, or the bone fixation component 57. The forks or paddles of the distractor are deployed against the opposing surfaces, such as (1) a native glenoid surface, the prepared glenoid surface 4, or the bone fixation component 10 and (2) the native humeral head surface, the prepared humeral surface 334, or the bone fixation component 57. The measuring involves reading the measurement indicator(s) at the reading indicator(s) 326, 328. Then a given sized articular component for the glenoid implant and/or the humeral implant is selected based on and corresponding to the measured distance.

Another method of determining the appropriate size of a humeral implant or glenoid implant or articulating components thereof is disclosed. The method comprises using a distraction measuring device 370 to measure the distance between opposing surfaces of the shoulder joint, such as (1) a native glenoid surface, the prepared glenoid surface 4, or the bone fixation component 10 and (2) a native humeral head surface, the prepared humeral surface 334 or the bone fixation component 57. The measuring involves placing the shaft 372 through the transhumeral tunnel 53 and placing the front 377 of the shaft against the native glenoid surface, the prepared glenoid surface 4, or the bone fixation component 10. Then reading the measurement indicator at the end 378 of the sleeve 376; or reading the measurement indicators between opposing surfaces of the shoulder joint, such as (1) a native glenoid surface, the prepared glenoid surface 4, or the bone fixation component 10 and (2) the native humeral head surface, the prepared humeral surface 334, or the bone fixation component 57. In some applications, when reading the measurement indicator at the end 378 of the sleeve 376, the distance between the opening 380 to the tunnel 53 and a native humeral head surface, the prepared humeral surface 334, or the bone fixation component 57 is subtracted from the reading of the measurement indicator at the end 378 of the sleeve 376 to determine the distance between opposing surfaces of the shoulder joint, such as (1) a native glenoid surface, the prepared glenoid surface 4, or the bone fixation component 10 and (2) a native humeral head surface, the prepared humeral surface 334, or the bone fixation component 57. Then a given sized articular component(s) for the glenoid implant and/or the humeral implant is selected based on and corresponding to the measured or calculated distance between opposing surfaces of the shoulder joint, such as (1) a native glenoid surface, the prepared glenoid surface 4, or the bone fixation component 10 and (2) the native humeral head surface, the prepared humeral surface 334, or the bone fixation component 57.

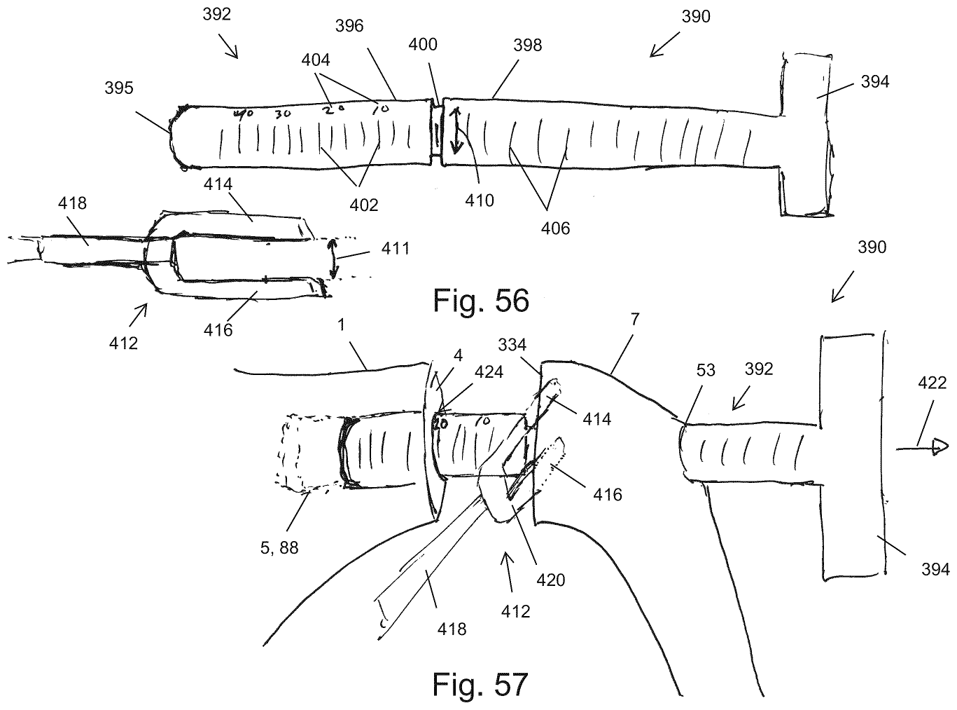

FIGS. 56 and 57 show a second embodiment measuring device 390. The device comprises a shaft 392. The shaft may comprise a transverse handle at a second end opposite a front end 395. The shaft 392 comprises a recessed portion 400 between a distal portion 396 and a proximal portion 398. A fork 412 comprises a plurality of spaced apart prongs 414, 416 joined to a handle 418. The prongs are spaced apart a width 411 that is sufficient to receive therein the recessed portion 400, the width 411 of the prongs is sufficiently narrow that the prongs will not fit around the distal portion 396 or the proximal portion 398. Therefore, the fork is configured to be received on the shaft 392 at the recess portion 400. The width 410 of the recess may be substantially the same as or smaller than the width 411 of the prongs so that the prongs may be received at the recess portion 400. The spacing of the prongs and the width of the shaft ensures that the fork is received in the recess on the shaft as it will not fit over the portions of the shaft that are adjacent to the recess. The portions 396, 398 of the shaft 392 comprise measurement indicators 402, 406. The measurement indicators may be arranged according to a scale of distance measurement along a length of the shaft, such as millimeters, centimeters, or inches (including fractions of an inch). One or more of the measurement indicators may comprise adjacent measurement numerals 404. In some embodiments, prongs 414, 416 may be configured to occupy all or substantially all of the length of the recess between the adjacent portion 396, 398 so as to provide a secure fit between the prongs and the shaft.

Another method of determining the appropriate size of a humeral implant or glenoid implant or articulating components thereof is disclosed. The method comprises using a measuring device 390 to measure the distance between opposing surfaces of the shoulder joint, such as (1) a native glenoid surface, the prepared glenoid surface 4, or the bone fixation component 10 and (2) a native humeral head surface, the prepared humeral surface 334 or the bone fixation component 57. The measuring involves placing the shaft 392 through the transhumeral tunnel 53 and placing the front 395 of the shaft into the glenoid cavity 5, which may include into the second portion 88 of the cavity 5. As the shaft 392 is in the transhumeral tunnel and in the glenoid cavity 5, the shaft can be used to align the humerus and the glenoid and the corresponding opposing surfaces thereof. The fork 412 is placed into the recess portion 400 of the shaft 392. The handle 394 of the device 390 is pulled/moved away from the glenoid, in the direction 422. This causes a first side 420 of the fork/prongs to contact a surface of the humerus, such as a native humeral head surface, the prepared humeral surface 334 or the bone fixation component 57. Then the measurement indicator 402 at the opening 424 to the glenoid cavity and/or at the native glenoid surface, the prepared glenoid surface 4, or the bone fixation component 10 will indicate a distance from the recess portion 400 thereto, and therefore, a distance between opposing surfaces of a shoulder joint, such as (1) a native glenoid surface, the prepared glenoid surface 4, or the bone fixation component 10 and (2) a native humeral head surface, the prepared humeral surface 334 or the bone fixation component 57. In some uses, a user can use the handle 394 to pull the shaft 392, and therefore the humerus via the fork, a distance sufficient to tension the soft tissue bridging the shoulder joint without dislocating the shoulder. Measurement can therefore be taken while soft tissue bridging the shoulder joint is tensioned by the device 390. In some embodiments, the fork 412 can be deployed on distractor 290, 332 in place of fork 304 and such distractor can be used together with the device 390 to tension the soft tissue bridging the shoulder joint and/or to measure the distance between opposing surfaces of the shoulder joint.

In some embodiments of the method of determining the appropriate size of a humeral implant or glenoid implant or articulating components disclosed herein, instead of or in addition to selecting a given sized articular component(s) for the glenoid implant and/or the humeral implant based on and corresponding to the measured and/or calculated distance between opposing surfaces of a shoulder joint, a trial articular component is selected based on the measured and/or calculated distance between opposing surfaces of a shoulder joint. The trial articular component is placed in the shoulder joint at the proximal humerus or at the glenoid without dislocating the shoulder joint. The shoulder joint is tested by moving the humerus. The trial articular component is removed. Thereafter an implant articular component may be implanted corresponding to the trial articular component or the steps of inserting, testing, removing are repeated until an appropriate sized or fitting trial articular component(s) is found, such as explained above.

In some embodiments, the method of determining the appropriate size of a humeral implant or glenoid implant or articulating components thereof, comprises using the device 370, and in particular the elongated shaft 372, in place through the transhumeral tunnel and into the glenoid cavity 5 to keep the glenoid and humerus in proper relationship, e.g. alignment. The alignment may be maintained using the shaft 372 while the soft tissue bridging the joint is tensioned, such as by distraction without dislocation. Measuring can be performed, with the distractor 290, 332, or with device 370, or with guidance from computer navigation, robotics, and/or augmented reality.

In some embodiments, before measuring a distance between opposing surfaces of the shoulder joint, such as (1) a native glenoid surface, the prepared glenoid surface 4, or the bone fixation component 10 and (2) a native humeral head surface, the prepared humeral surface 334, or the bone fixation component 57, the soft tissue bridging the shoulder joint is tensioned such as by applying distraction without dislocating the shoulder joint. Such distraction may be applied by a distractor 134, 290, 332, by an instrument 65, and/or by manually pulling the humerus away from the glenoid. Therefore the soft tissue is tensioned during measuring.

In some embodiments, the distance between the glenoid and humerus, with or without trial or implant components in place, is determined using optical tracking and computer navigation, robotic, and/or augmented reality guidance.

The methods of determining and/or selecting the appropriate size of a humeral implant or glenoid implant or articulating components thereof disclosed herein may be used as a part of partial or total shoulder replacement surgery, wherein the determined/selected humeral implant and/or glenoid implant or components thereof is/are implanted in the shoulder joint, such as at the proximal humerus and/or the glenoid. At least one of the novel aspects of these methods is that the methods may be employed without dislocating the shoulder joint and optionally without transversely cutting a rotator cuff.

The glenoid implants 30, 39, 51, 71, 90 provide a stable, long lasting manner of engaging an implant in a patient's shoulder utilizing an improved junction between its articulating portion and the implant's bone fixation portion. This junction makes it possible to replace the articular portion without disturbing the implant's connection to the glenoid bone and exchange a worn or failed articular component or convert between an anatomic relatively concave glenoid articular component or reverse convex glenoid articular component as needed, as in the case of rotator cuff failure, for example.

In some embodiments, the glenoid implants 30, 39, 51, 71, 90 and the associated methods disclosed herein seek to provide improved surgical techniques and seek to restore shoulder function and to relieve pain.

The glenoid implants 30, 39, 51, 71, 90 can be used in repairing and reconstructing injured shoulder joints. In some embodiments, surgery outcomes of joint replacement surgery can be improved by providing a glenoid implant 30, 39, 51, 71, 90 having an articulating portion which can be replaced, if it becomes worn, or converted to a reverse total shoulder arthroplasty, or back to an anatomic configuration from total shoulder arthroplasty, if that should be necessary or desirable, without disturbing the implant's connection to the glenoid bone.

In some embodiments, glenoid implants 30, 39, 51, 71, 90 disclosed herein for shoulder replacement surgery can be implanted with conventional or minimally invasive techniques or without dislocating the shoulder joint. The methods involving trial components described herein in connection with FIGS. 32-36, 40, and/or 50 that are performed without dislocating the shoulder joint, and optionally without transversely cutting the rotator cuff can also be employed with humeral and/or glenoid implants, including components thereof. Therefore, insertion and fixation of humeral and glenoid implants, including components thereof, at the humerus and/or glenoid can be performed without dislocating the shoulder joint, and may be done under distraction and optionally without transversely cutting the rotator cuff.

In some embodiments, the glenoid implant 30, 39, 71, 90, comprises a bone fixation portion and an articulating wear-resistant portion along with an intermediate coupling portion which supplies a durable but detachable coupling between the bone fixation and articulating portions. In some embodiments, a post 52, 70, 76, 92 also assists in supplying a durable but detachable coupling between the bone fixation and articulating portions. In some embodiments, such as glenoid implant 51, the post supplies a durable but detachable coupling between the bone fixation and articulating portions in place of or as a coupling portion. In some embodiments, the post can also assist with bony fixation to the second portion 88 of bone cavity 5, and the fixation may be reversable or intended to be irreversible to the bony cavity. depending on surface properties of the post. The surface properties of the post may be smooth with no-adhering properties or threaded or with a bone ingrowth adherent surface along all or a portion of the length and/or circumference of the post.

In some embodiments, a friction fit or press fit connection, such as a conical press fit or morse taper connection, is provided between one or more or all of: the bone fixation portion 10 and the coupling portion 24, 24b, 24c; the coupling portion 24, 24b, 24c and the articulating portion 20, 20b, 20d, 20e; the post 52 and the articulating portion 20c and/or portion 88 of cavity 5; the post 70, 76, 92 and the coupling portion 24c and/or portion 88 of cavity 5; the first post 92 and the second post 94, and/or the second post 94 and the coupling portion 20d to provide a durable but detachable connection or a long-lasting but disengageable connection between those friction fit/press fitted components or portions and also between the bone fixation portion and the articulating portion. Therefore, the relevant connecting surfaces may be a proper dimension and material to allow press fit connection or a conical press fit or morse taper connection.

In some embodiments, threaded connections could be provided in addition to or as a substitute for press fit connections between one or more or all of: the bone fixation portion 10 and the coupling portion 24, 24b, 24c; the coupling portion 24, 24b, 24c and the articulating portion 20, 20b, 20d, 20e; the post 52 and the articulating portion 20c and/or portion 88 of cavity 5; the post 70, 76, 92 and the coupling portion 24c and/or portion 88 of cavity 5; the first post 92 and the second post 94, and/or the second post 94 and the coupling portion 20d to provide a durable but detachable connection or a long-lasting but disengageable connection between those threaded components or portions and also between the bone fixation portion and the articulating portion.

In some embodiments, the glenoid implant 30, 39, 51, 71, 90 comprises a bone fixation portion having geometry which affords long-lasting fixation of that portion to a glenoid bone and a durable and separable connection to a coupling portion and/or a post of the implant engaged on an articulating wear-resistant portion of the implant.

In some embodiments, the glenoid implant has a bone fixation portion which resides on the glenoid surface (onlay) and also in the glenoid vault (inlay) of a scapular bone.

In some embodiments, the glenoid implant is arranged for correcting pathological conditions regarding offset, version, inclination, bone loss, bone fractures, medialization, joint instability and rotator cuff deficiency.

In some embodiments, the glenoid implant 30, 39, 51, 71, 90 comprises an articulating portion 20, 20b, 20c, 20d, 20e that is coupled to a firmly fixed bone fixation portion 10 and exchangeable with another articulating portion 20, 20b, 20c, 20d, 20e without disturbing any previous fixation of the bone fixation portion.

In some embodiments, the glenoid implant 30, 39, 51, 71, 90 comprises an articulating portion 20, 20b, 20c, 20d, 20e which can be converted to a different articulating portion 20, 20b, 20c, 20d, 20e of alternative dimensions, material, and purpose while the bone fixation portion 10 of the implant remains well fixed.

In some embodiments, the glenoid implant 30, 39, 51, 71, 90 comprises an articulating portion 20, 20b, 20c, 20d, 20e, disposed in an anatomic total shoulder arthroplasty arranged for conversion to a reverse total shoulder arthroplasty and vice versa.

From the foregoing, it will be observed that numerous variations and modifications may be affected without departing from the spirit and scope of the invention. It is to be understood that no limitation with respect to the specific apparatus illustrated herein is intended or should be inferred. For example, one or more component embodiments may be combined, modified, removed, or supplemented to form further embodiments within the scope of the invention. Further, steps could be added or removed from the processes described. Therefore, other embodiments and implementations are within the scope of the invention. It is therefore intended that the foregoing detailed description be regarded as illustrative rather than limiting, and that it be understood that it is the following claims, including all equivalents, that are intended to define the spirit and scope of this invention.

The invention claimed is:

1. A glenoid implant comprising:
a bone fixation portion comprising a first side, an articular side, a first protruding element on the first side, and a front surface on the articular side, the bone fixation portion defines a first socket open on the articular side adjacent the front surface, the first protruding element arranged to engage a bone surface inside a glenoid cavity;
an articulating portion comprising a wear-resistant articulating surface arranged for sliding contact upon an articulating humeral portion of a humerus, a rear side, a second socket open on the rear side, and a rear surface adjacent the second socket; and
a coupling portion arranged to connect the bone fixation portion and the articulating portion, the coupling portion is engaged with the second socket of the articulating portion and the coupling portion is engaged with the bone fixation portion in the first socket to form a stable long-lasting but disengageable connection between the bone fixation portion and the articulating portion, the front surface of the bone fixation portion is behind the rear surface of the articulating portion.

2. The glenoid implant of claim 1, wherein the coupling portion comprises a second protruding element on a second side of the coupling portion and a first portion on a first side of the coupling portion, the first portion is engaged with the second socket of the articulating portion and the second protruding element is engaged with the bone fixation portion in the first socket to form the stable long-lasting but disengageable connection between the bone fixation portion and the articulating portion.

3. The glenoid implant of claim 2, wherein the first portion is in front the second protruding element along a length of the coupling portion, the first portion comprises a width, transverse to the length, that is greater than a width, transverse to the length, of the second protruding element.

4. The glenoid implant of claim 2, wherein the second protruding element extends from the first portion.

5. The glenoid implant of claim 2, wherein the second protruding element and the first portion are each cylindrical, and the first portion comprises a diameter that is larger than a diameter of the second protruding element.

6. The glenoid implant of claim 1, wherein the coupling portion is sized to friction fit to the bone fixation portion in the first socket and is sized to friction fit to the articulating portion in the second socket.

7. The glenoid implant of claim 2, wherein the first portion of the coupling portion is sized to fiction fit to the bone fixation portion in the first socket.

8. The glenoid implant of claim 2, wherein the first portion comprises a length that matches a depth of the first socket.

9. The glenoid implant of claim 2, wherein a depth of the first socket is greater than a length of the first portion.

10. The glenoid implant of claim 1, wherein the coupling portion is a post.

11. The glenoid implant of claim 10, wherein the post is cylindrical.

12. The glenoid implant of claim 10, wherein
the first socket is a first cannulation extending from a first aperture on the first protruding element on the first side to a second aperture on the articular side; and,
the post configured to engage the bone fixation portion at the first cannulation.

13. The glenoid implant of claim 12, wherein the second socket of the articulating portion is a second cannulation extending through the articulating portion from a third aperture on the articulating surface to a fourth aperture on the rear side, and the post configured to engage the articulating portion at the second cannulation.

14. The glenoid implant of claim 10, wherein the post is configured to engage the bone surface or a second bone surface at the glenoid cavity.

15. The glenoid implant of claim 10, wherein the second socket of the articulating portion is a second cannulation extending through the articulating portion from a third aperture on the articulating surface to a fourth aperture on the rear side, and the post configured to engage the articulating portion at the second cannulation.

16. The glenoid implant of claim 15, wherein a front of the post forms an articulating surface portion together with the articulating surface when the post is engaged at the second cannulation.

17. The glenoid implant of claim 10, wherein the post comprises a plurality of threads at an end of the post for reversibly engaging a glenoid bone, or a smooth non-bone-adherent surface at the end of the post for reversibly engaging the glenoid bone, or a bony ingrowth surface for engaging the glenoid bone.

18. The glenoid implant of claim 1, comprising a post; and,
the first socket is a first cannulation, and
the coupling portion comprises a second protruding element on a first side of the coupling portion, the coupling portion defines a second cannulation extending through the coupling portion from a third aperture on a second side of the coupling portion to a fourth aperture on the first side of the coupling portion, the post is engaged with the coupling portion in the second cannulation, and the second protruding element is engaged with the bone fixation portion in the first cannulation.

19. The glenoid implant of claim 18, where the post extends through the coupling portion and through the bone fixation portion and is configured to engage the bone surface or a second bone surface at the glenoid cavity.

20. The glenoid implant of claim 18, wherein the post is a first post, and wherein the glenoid implant comprises a second post, and,
the articulating portion comprises a third cannulation extending through the articulating portion from a fifth aperture on the articulating surface to a sixth aperture on the rear side of the articulating portion;
the first post comprises a fourth cannulation extending from a seventh aperture in a front side of the first post; and,
the second post is engageable with the articulating portion at the third cannulation and the first post at the fourth cannulation to form a connection between the articulating portion and the first post.

21. The glenoid implant of claim 20, wherein the first post comprises a threaded perimeter and the second post comprises a threaded perimeter.

22. A method of shoulder replacement, comprising a plurality of steps of:
placing a bone fixation portion comprising a first cannulation at a prepared glenoid cavity;
placing an articulating portion comprising a second cannulation at the bone fixation portion; and,
inserting a post transhumerally and engaging the articulating portion at the second cannulation and the bone fixation portion at the first cannulation to form a stable long-lasting but disengageable connection between the bone fixation portion and the articulating portion.

23. The method of claim 22, wherein the step of inserting comprises inserting the post into the glenoid cavity.

24. A method of shoulder replacement, comprising a plurality of steps of:
fixing a bone fixation portion comprising a first cannulation at a prepared glenoid cavity;
fixing a coupling portion at the first cannulation;
fixing an articulating portion at the coupling portion within a recess of the articulating portion.
